SEQUENCE LISTING (329 sequences)

SEQ ID NO: 1 - DNA, 25 bp, Homo sapiens
cacagtggagcgaattcctttggaa

SEQ ID NO: 2 - DNA, 25 bp, Homo sapiens
cgcaaagtgtgtaacggaataggta

SEQ ID NO: 3 - DNA, 25 bp, Homo sapiens
ggatcccagaaggtgagaaagttaa

SEQ ID NO: 4 - DNA, 25 bp, Homo sapiens
cgcagcatgtcaagatcacagattt

SEQ ID NO: 5 - DNA, 25 bp, Homo sapiens
cagaaggaggcaaagtgcctatcaa

SEQ ID NO: 6 - DNA, 25 bp, Homo sapiens
gcctatcaagtggatggcattggaa

SEQ ID NO: 7 - DNA, 25 bp, Homo sapiens
ccaagtcctacagactccaacttct

SEQ ID NO: 8 - DNA, 25 bp, Homo sapiens
tctctgagtgcaaccagcaacaatt

SEQ ID NO: 9 - DNA, 25 bp, Homo sapiens
cagcttcttgcagcgatacagctca

SEQ ID NO: 10 - DNA, 25 bp, Homo sapiens
gaagccaagccaaatggcatcttta

SEQ ID NO: 11 - RNA, 25 nt, Artificial Sequence
  Description of Artificial Sequence Synthetic oligonucleotide
cacaguggagcgaauuccuuuggaa

SEQ ID NO: 12 - RNA, 25 nt, Artificial Sequence
  Description of Artificial Sequence Synthetic oligonucleotide
uuccaaaggaauucgcuccacugug

SEQ ID NO: 13 - RNA, 25 nt, Artificial Sequence
  Description of Artificial Sequence Synthetic oligonucleotide
cgcaaaguguguaacggaauaggua

SEQ ID NO: 14 - RNA, 25 nt, Artificial Sequence
  Description of Artificial Sequence Synthetic oligonucleotide
uaccuauuccguuacacacuuugcg

SEQ ID NO: 15 - RNA, 25 nt, Artificial Sequence
  Description of Artificial Sequence Synthetic oligonucleotide
ggaucccagaaggugagaaaguuaa

SEQ ID NO: 16 - RNA, 25 nt, Artificial Sequence
  Description of Artificial Sequence Synthetic oligonucleotide
uuaacuuucucaccuucugggaucc

SEQ ID NO: 17 - RNA, 25 nt, Artificial Sequence
  Description of Artificial Sequence Synthetic oligonucleotide
cgcagcaugucaagaucacagauuu

SEQ ID NO: 18 - RNA, 25 nt, Artificial Sequence
  Description of Artificial Sequence Synthetic oligonucleotide
aaaucugugaucuugacaugcugcg

SEQ ID NO: 19 - RNA, 25 nt, Artificial Sequence
  Description of Artificial Sequence Synthetic oligonucleotide
ccaaguccuacagacuccaacuucu

SEQ ID NO: 20 - RNA, 25 nt, Artificial Sequence
  Description of Artificial Sequence Synthetic oligonucleotide
agaaguuggagucuguaggacuugg

SEQ ID NO: 21 - DNA, 25 bp, Homo sapiens
tgccaaggcacgagtaacaagctca

SEQ ID NO: 22 - DNA, 25 bp, Homo sapiens
gcacgagtaacaagctcacgcagtt

SEQ ID NO: 23 - DNA, 25 bp, Homo sapiens
tggaaattacctatgtgcagaggaa

SEQ ID NO: 24 - DNA, 25 bp, Homo sapiens
ggaaattacctatgtgcagaggaat

SEQ ID NO: 25 - DNA, 25 bp, Homo sapiens
gaaattacctatgtgcagaggaatt

SEQ ID NO: 26 - DNA, 25 bp, Homo sapiens
tacctatgtgcagaggaattatgat

SEQ ID NO: 27 - DNA, 25 bp, Homo sapiens
cctatgtgcagaggaattatgatct

SEQ ID NO: 28 - DNA, 25 bp, Homo sapiens
tgcagaggaattatgatctttcctt

SEQ ID NO: 29 - DNA, 25 bp, Homo sapiens
cagaggaattatgatctttccttct

SEQ ID NO: 30 - DNA, 25 bp, Homo sapiens
tcctatgccttagcagtcttatcta

SEQ ID NO: 31 - DNA, 25 bp, Homo sapiens
cctatgccttagcagtcttatctaa

SEQ ID NO: 32 - DNA, 25 bp, Homo sapiens
ccttagcagtcttatctaactatga

SEQ ID NO: 33 - DNA, 25 bp, Homo sapiens
gggacatagtcagcagtgactttct

SEQ ID NO: 34 - DNA, 25 bp, Homo sapiens
tagtcagcagtgactttctcagcaa

SEQ ID NO: 35 - DNA, 25 bp, Homo sapiens
cccgtaattatgtggtgacagatca

SEQ ID NO: 36 - DNA, 25 bp, Homo sapiens
ccgtaattatgtggtgacagatcac

SEQ ID NO: 37 - DNA, 25 bp, Homo sapiens
cgtccgcaagtgtaagaagtgcgaa

SEQ ID NO: 38 - DNA, 25 bp, Homo sapiens
caagtgtaagaagtgcgaagggcct

SEQ ID NO: 39 - DNA, 25 bp, Homo sapiens
ccttgccgcaaagtgtgtaacggaa

SEQ ID NO: 40 - DNA, 25 bp, Homo sapiens
ccgcaaagtgtgtaacggaataggt

SEQ ID NO: 41 - DNA, 25 bp, Homo sapiens
cgcaaagtgtgtaacggaataggta

SEQ ID NO: 42 - DNA, 25 bp, Homo sapiens
gcaaagtgtgtaacggaataggtat

SEQ ID NO: 43 - DNA, 25 bp, Homo sapiens
caaagtgtgtaacggaataggtatt

SEQ ID NO: 44 - DNA, 25 bp, Homo sapiens
cactctccataaatgctacgaatat

SEQ ID NO: 45 - DNA, 25 bp, Homo sapiens
cctctggatccacaggaactggata

SEQ ID NO: 46 - DNA, 25 bp, Homo sapiens
tggatccacaggaactggatattct

SEQ ID NO: 47 - DNA, 25 bp, Homo sapiens
tccatgcctttgagaacctagaaat

SEQ ID NO: 48 - DNA, 25 bp, Homo sapiens
caggaccaagcaacatggtcagttt

SEQ ID NO: 49 - DNA, 25 bp, Homo sapiens
tctcttgcagtcgtcagcctgaaca

SEQ ID NO: 50 - DNA, 25 bp, Homo sapiens
catccttgggattacgctccctcaa

SEQ ID NO: 51 - DNA, 25 bp, Homo sapiens
ccctcaaggagataagtgatggaga

SEQ ID NO: 52 - DNA, 25 bp, Homo sapiens
cctcaaggagataagtgatggagat

SEQ ID NO: 53 - DNA, 25 bp, Homo sapiens
tcaaggagataagtgatggagatgt

SEQ ID NO: 54 - DNA, 25 bp, Homo sapiens
aggagataagtgatggagatgtgat

SEQ ID NO: 55 - DNA, 25 bp, Homo sapiens
ggagataagtgatggagatgtgata

SEQ ID NO: 56 - DNA, 25 bp, Homo sapiens
gagataagtgatggagatgtgataa

SEQ ID NO: 57 - DNA, 25 bp, Homo sapiens
ccaagggagtttgtggagaactctg

SEQ ID NO: 58 - DNA, 25 bp, Homo sapiens
gagtttgtggagaactctgagtgca

SEQ ID NO: 59 - DNA, 25 bp, Homo sapiens
cagacaactgtatccagtgtgccca

SEQ ID NO: 60 - DNA, 25 bp, Homo sapiens
tgtatccagtgtgcccactacattg

SEQ ID NO: 61 - DNA, 25 bp, Homo sapiens
catccaaactgcacctacggatgca

SEQ ID NO: 62 - DNA, 25 bp, Homo sapiens
gcacggtgtataagggactctggat

SEQ ID NO: 63 - DNA, 25 bp, Homo sapiens
ccgtcgctatcaaggaattaagaga

SEQ ID NO: 64 - DNA, 25 bp, Homo sapiens
cgtcgctatcaaggaattaagagaa

SEQ ID NO: 65 - DNA, 25 bp, Homo sapiens
cgctatcaaggaattaagagaagca

SEQ ID NO: 66 - DNA, 25 bp, Homo sapiens
caaggaattaagagaagcaacatct

SEQ ID NO: 67 - DNA, 25 bp, Homo sapiens
gaaatcctcgatgaagcctacgtga

SEQ ID NO: 68 - DNA, 25 bp, Homo sapiens
ccgcagcatgtcaagatcacagatt

SEQ ID NO: 69 - DNA, 25 bp, Homo sapiens
cgcagcatgtcaagatcacagattt

SEQ ID NO: 70 - DNA, 25 bp, Homo sapiens
catgcagaaggaggcaaagtgccta

SEQ ID NO: 71 - DNA, 25 bp, Homo sapiens
cagaaggaggcaaagtgcctatcaa

SEQ ID NO: 72 - DNA, 25 bp, Homo sapiens
aagtgcctatcaagtggatggcatt

SEQ ID NO: 73 - DNA, 25 bp, Homo sapiens
gcctatcaagtggatggcattggaa

SEQ ID NO: 74 - DNA, 25 bp, Homo sapiens
cctatcaagtggatggcattggaat

SEQ ID NO: 75 - DNA, 25 bp, Homo sapiens
caagtggatggcattggaatcaatt

SEQ ID NO: 76 - DNA, 25 bp, Homo sapiens
cagccacccatatgtaccatcgatg

SEQ ID NO: 77 - DNA, 25 bp, Homo sapiens
cacccatatgtaccatcgatgtcta

SEQ ID NO: 78 - DNA, 25 bp, Homo sapiens
tgtaccatcgatgtctacatgatca

SEQ ID NO: 79 - DNA, 25 bp, Homo sapiens
tagacgcagatagtcgcccaaagtt

SEQ ID NO: 80 - DNA, 25 bp, Homo sapiens
ccaagtcctacagactccaacttct

SEQ ID NO: 81 - DNA, 25 bp, Homo sapiens
caagtcctacagactccaacttcta

SEQ ID NO: 82 - DNA, 25 bp, Homo sapiens
actccaacttctaccgtgccctgat

SEQ ID NO: 83 - DNA, 25 bp, Homo sapiens
tctctgagtgcaaccagcaacaatt

SEQ ID NO: 84 - DNA, 25 bp, Homo sapiens
caattccaccgtggcttgcattgat

SEQ ID NO: 85 - DNA, 25 bp, Homo sapiens
ccaccgtggcttgcattgatagaaa

SEQ ID NO: 86 - DNA, 25 bp, Homo sapiens
caccgtggcttgcattgatagaaat

SEQ ID NO: 87 - DNA, 25 bp, Homo sapiens
tgcattgatagaaatgggctgcaaa

SEQ ID NO: 88 - DNA, 25 bp, Homo sapiens
gcattgatagaaatgggctgcaaag

SEQ ID NO: 89 - DNA, 25 bp, Homo sapiens
cagcttcttgcagcgatacagctca

SEQ ID NO: 90 - DNA, 25 bp, Homo sapiens
ccagtgcctgaatacataaaccagt

SEQ ID NO: 91 - DNA, 25 bp, Homo sapiens
ccacctgtgtcaacagcacattcga

SEQ ID NO: 92 - DNA, 25 bp, Homo sapiens
gcccagaaaggcagccaccaaatta

SEQ ID NO: 93 - DNA, 25 bp, Homo sapiens
cccagaaaggcagccaccaaattag

SEQ ID NO: 94 - DNA, 25 bp, Homo sapiens
ggaagccaagccaaatggcatcttt

SEQ ID NO: 95 - DNA, 25 bp, Homo sapiens
gaagccaagccaaatggcatcttta

SEQ ID NO: 96 - DNA, 25 bp, Homo sapiens
aagccaagccaaatggcatctttaa

SEQ ID NO: 97 - DNA, 25 bp, Homo sapiens
tggcatctttaagggctccacagct

SEQ ID NO: 98 - DNA, 25 bp, Homo sapiens
catctttaagggctccacagctgaa

SEQ ID NO: 99 - DNA, 25 bp, Homo sapiens
ccacggaggatagtatgagccctaa

SEQ ID NO: 100 - DNA, 25 bp, Homo sapiens
cacggaggatagtatgagccctaaa

SEQ ID NO: 101 - DNA, 25 bp, Homo sapiens
tacagaaacgcatccagcaagaata

SEQ ID NO: 102 - DNA, 25 bp, Homo sapiens
tgatggaccagtggtttccagtcat

SEQ ID NO: 103 - DNA, 25 bp, Homo sapiens
cagtggtttccagtcatgagcgtta

SEQ ID NO: 104 - DNA, 25 bp, Homo sapiens
cagcaagagaggatgacacatcaaa

SEQ ID NO: 105 - DNA, 25 bp, Homo sapiens
ccagcccacattggattcatcagca

SEQ ID NO: 106 - DNA, 25 bp, Homo sapiens
cagcccacattggattcatcagcat

SEQ ID NO: 107 - DNA, 25 bp, Homo sapiens
gcccacattggattcatcagcattt

SEQ ID NO: 108 - DNA, 25 bp, Homo sapiens
ccacagctgagaatgtggaatacct

SEQ ID NO: 109 - DNA, 25 bp, Homo sapiens
cacagctgagaatgtggaataccta

SEQ ID NO: 110 - DNA, 25 bp, Homo sapiens
tctcctaatttgaggctcagatgaa

SEQ ID NO: 111 - DNA, 25 bp, Homo sapiens
gaggctcagatgaaatgcatcaggt

SEQ ID NO: 112 - DNA, 25 bp, Homo sapiens
caggtgcgaatgacagtagcattat

SEQ ID NO: 113 - DNA, 25 bp, Homo sapiens
gcgaatgacagtagcattatgagta

SEQ ID NO: 114 - DNA, 25 bp, Homo sapiens
cagtagcattatgagtagtgtggaa

SEQ ID NO: 115 - DNA, 25 bp, Homo sapiens
gcattatgagtagtgtggaattcag

SEQ ID NO: 116 - DNA, 25 bp, Homo sapiens
agtagtgtggaattcaggtagtaaa

SEQ ID NO: 117 - DNA, 25 bp, Homo sapiens
tgtgccctgtaacctgactggttaa

SEQ ID NO: 118 - DNA, 25 bp, Homo sapiens
cctgactggttaacagcagtccttt

SEQ ID NO: 119 - DNA, 25 bp, Homo sapiens
gactggttaacagcagtcctttgta

SEQ ID NO: 120 - DNA, 25 bp, Homo sapiens
cagcagtcctttgtaaacagtgttt

SEQ ID NO: 121 - DNA, 25 bp, Homo sapiens
cagcctacagttatgttcagtcaca

SEQ ID NO: 122 - RNA, 25 nt, Artificial Sequence
  Description of Artificial Sequence Synthetic oligonucleotide
ugccaaggcacgaguaacaagcuca

SEQ ID NO: 123 - RNA, 25 nt, Artificial Sequence
  Description of Artificial Sequence Synthetic oligonucleotide
ugagcuuguuacucgugccuuggca

SEQ ID NO: 124 - RNA, 25 nt, Artificial Sequence
  Description of Artificial Sequence Synthetic oligonucleotide
gcacgaguaacaagcucacgcaguu

SEQ ID NO: 125 - RNA, 25 nt, Artificial Sequence
  Description of Artificial Sequence Synthetic oligonucleotide
aacugcgugagcuuguuacucgugc

SEQ ID NO: 126 - RNA, 25 nt, Artificial Sequence
  Description of Artificial Sequence Synthetic oligonucleotide
uggaaauuaccuaugugcagaggaa

SEQ ID NO: 127 - RNA, 25 nt, Artificial Sequence
  Description of Artificial Sequence Synthetic oligonucleotide
uuccucugcacauagguaauuucca

SEQ ID NO: 128 - RNA, 25 nt, Artificial Sequence
  Description of Artificial Sequence Synthetic oligonucleotide
ggaaauuaccuaugugcagaggaau

SEQ ID NO: 129 - RNA, 25 nt, Artificial Sequence
  Description of Artificial Sequence Synthetic oligonucleotide
auuccucugcacauagguaauuucc

SEQ ID NO: 130 - RNA, 25 nt, Artificial Sequence
  Description of Artificial Sequence Synthetic oligonucleotide
gaaauuaccuaugugcagaggaauu

SEQ ID NO: 131 - RNA, 25 nt, Artificial Sequence
  Description of Artificial Sequence Synthetic oligonucleotide
aauuccucugcacauagguaauuuc

SEQ ID NO: 132 - RNA, 25 nt, Artificial Sequence
  Description of Artificial Sequence Synthetic oligonucleotide
uaccuaugugcagaggaauuaugau

SEQ ID NO: 133 - RNA, 25 nt, Artificial Sequence
  Description of Artificial Sequence Synthetic oligonucleotide
aucauaauuccucugcacauaggua

SEQ ID NO: 134 - RNA, 25 nt, Artificial Sequence
  Description of Artificial Sequence Synthetic oligonucleotide
ccuaugugcagaggaauuaugaucu

SEQ ID NO: 135 - RNA, 25 nt, Artificial Sequence
  Description of Artificial Sequence Synthetic oligonucleotide
agaucauaauuccucugcacauagg

SEQ ID NO: 136 - RNA, 25 nt, Artificial Sequence
  Description of Artificial Sequence Synthetic oligonucleotide
ugcagaggaauuaugaucuuuccuu

SEQ ID NO: 137 - RNA, 25 nt, Artificial Sequence
  Description of Artificial Sequence Synthetic oligonucleotide
aaggaaagaucauaauuccucugca

SEQ ID NO: 138 - RNA, 25 nt, Artificial Sequence
  Description of Artificial Sequence Synthetic oligonucleotide
cagaggaauuaugaucuuuccuucu

SEQ ID NO: 139 - RNA, 25 nt, Artificial Sequence
  Description of Artificial Sequence Synthetic oligonucleotide
agaaggaaagaucauaauuccucug

SEQ ID NO: 140 - RNA, 25 nt, Artificial Sequence
  Description of Artificial Sequence Synthetic oligonucleotide
uccuaugccuuagcagucuuaucua

SEQ ID NO: 141 - RNA, 25 nt, Artificial Sequence
  Description of Artificial Sequence Synthetic oligonucleotide
uagauaagacugcuaaggcauagga

SEQ ID NO: 142 - RNA, 25 nt, Artificial Sequence
  Description of Artificial Sequence Synthetic oligonucleotide
ccuaugccuuagcagucuuaucuaa

SEQ ID NO: 143 - RNA, 25 nt, Artificial Sequence
  Description of Artificial Sequence Synthetic oligonucleotide
uuagauaagacugcuaaggcauagg

SEQ ID NO: 144 - RNA, 25 nt, Artificial Sequence
  Description of Artificial Sequence Synthetic oligonucleotide
ccuuagcagucuuaucuaacuauga

SEQ ID NO: 145 - RNA, 25 nt, Artificial Sequence
  Description of Artificial Sequence Synthetic oligonucleotide
ucauaguuagauaagacugcuaagg

SEQ ID NO: 146 - RNA, 25 nt, Artificial Sequence
  Description of Artificial Sequence Synthetic oligonucleotide
gggacauagucagcagugacuuucu

SEQ ID NO: 147 - RNA, 25 nt, Artificial Sequence
  Description of Artificial Sequence Synthetic oligonucleotide
agaaagucacugcugacuauguccc

SEQ ID NO: 148 - RNA, 25 nt, Artificial Sequence
  Description of Artificial Sequence Synthetic oligonucleotide
uagucagcagugacuuucucagcaa

SEQ ID NO: 149 - RNA, 25 nt, Artificial Sequence
  Description of Artificial Sequence Synthetic oligonucleotide
uugcugagaaagucacugcugacua

SEQ ID NO: 150 - RNA, 25 nt, Artificial Sequence
  Description of Artificial Sequence Synthetic oligonucleotide
cccguaauuauguggugacagauca

SEQ ID NO: 151 - RNA, 25 nt, Artificial Sequence
  Description of Artificial Sequence Synthetic oligonucleotide
ugaucugucaccacauaauuacggg

SEQ ID NO: 152 - RNA, 25 nt, Artificial Sequence
  Description of Artificial Sequence Synthetic oligonucleotide
ccguaauuauguggugacagaucac

SEQ ID NO: 153 - RNA, 25 nt, Artificial Sequence
  Description of Artificial Sequence Synthetic oligonucleotide
gugaucugucaccacauaauuacgg

SEQ ID NO: 154 - RNA, 25 nt, Artificial Sequence
  Description of Artificial Sequence Synthetic oligonucleotide
cguccgcaaguguaagaagugcgaa

SEQ ID NO: 155 - RNA, 25 nt, Artificial Sequence
  Description of Artificial Sequence Synthetic oligonucleotide
uucgcacuucuuacacuugcggacg

SEQ ID NO: 156 - RNA, 25 nt, Artificial Sequence
  Description of Artificial Sequence Synthetic oligonucleotide
caaguguaagaagugcgaagggccu

SEQ ID NO: 157 - RNA, 25 nt, Artificial Sequence
  Description of Artificial Sequence Synthetic oligonucleotide
aggcccuucgcacuucuuacacuug

SEQ ID NO: 158 - RNA, 25 nt, Artificial Sequence
  Description of Artificial Sequence Synthetic oligonucleotide
ccuugccgcaaaguguguaacggaa

SEQ ID NO: 159 - RNA, 25 nt, Artificial Sequence
  Description of Artificial Sequence Synthetic oligonucleotide
uuccguuacacacuuugcggcaagg

SEQ ID NO: 160 - RNA, 25 nt, Artificial Sequence
  Description of Artificial Sequence Synthetic oligonucleotide
ccgcaaaguguguaacggaauaggu

SEQ ID NO: 161 - RNA, 25 nt, Artificial Sequence
  Description of Artificial Sequence Synthetic oligonucleotide
accuauuccguuacacacuuugcgg

SEQ ID NO: 162 - RNA, 25 nt, Artificial Sequence
  Description of Artificial Sequence Synthetic oligonucleotide
cgcaaaguguguaacggaauaggua

SEQ ID NO: 163 - RNA, 25 nt, Artificial Sequence
  Description of Artificial Sequence Synthetic oligonucleotide
uaccuauuccguuacacacuuugcg

SEQ ID NO: 164 - RNA, 25 nt, Artificial Sequence
  Description of Artificial Sequence Synthetic oligonucleotide
gcaaaguguguaacggaauagguau

SEQ ID NO: 165 - RNA, 25 nt, Artificial Sequence
  Description of Artificial Sequence Synthetic oligonucleotide
auaccuauuccguuacacacuuugc

SEQ ID NO: 166 - RNA, 25 nt, Artificial Sequence
  Description of Artificial Sequence Synthetic oligonucleotide
caaaguguguaacggaauagguauu

SEQ ID NO: 167 - RNA, 25 nt, Artificial Sequence
  Description of Artificial Sequence Synthetic oligonucleotide
aauaccuauuccguuacacacuuug

SEQ ID NO: 168 - RNA, 25 nt, Artificial Sequence
  Description of Artificial Sequence Synthetic oligonucleotide
cacucuccauaaaugcuacgaauau

SEQ ID NO: 169 - RNA, 25 nt, Artificial Sequence
  Description of Artificial Sequence Synthetic oligonucleotide
auauucguagcauuuauggagagug

SEQ ID NO: 170 - RNA, 25 nt, Artificial Sequence
  Description of Artificial Sequence Synthetic oligonucleotide
ccucuggauccacaggaacuggaua

SEQ ID NO: 171 - RNA, 25 nt, Artificial Sequence
  Description of Artificial Sequence Synthetic oligonucleotide
uauccaguuccuguggauccagagg

SEQ ID NO: 172 - RNA, 25 nt, Artificial Sequence
  Description of Artificial Sequence Synthetic oligonucleotide
uggauccacaggaacuggauauucu

SEQ ID NO: 173 - RNA, 25 nt, Artificial Sequence
  Description of Artificial Sequence Synthetic oligonucleotide
agaauauccaguuccuguggaucca

SEQ ID NO: 174 - RNA, 25 nt, Artificial Sequence
  Description of Artificial Sequence Synthetic oligonucleotide
uccaugccuuugagaaccuagaaau

SEQ ID NO: 175 - RNA, 25 nt, Artificial Sequence
  Description of Artificial Sequence Synthetic oligonucleotide
auuucuagguucucaaaggcaugga

SEQ ID NO: 176 - RNA, 25 nt, Artificial Sequence
  Description of Artificial Sequence Synthetic oligonucleotide
caggaccaagcaacauggucaguuu

SEQ ID NO: 177 - RNA, 25 nt, Artificial Sequence
  Description of Artificial Sequence Synthetic oligonucleotide
aaacugaccauguugcuugguccug

SEQ ID NO: 178 - RNA, 25 nt, Artificial Sequence
  Description of Artificial Sequence Synthetic oligonucleotide
ucucuugcagucgucagccugaaca

SEQ ID NO: 179 - RNA, 25 nt, Artificial Sequence
  Description of Artificial Sequence Synthetic oligonucleotide
uguucaggcugacgacugcaagaga

SEQ ID NO: 180 - RNA, 25 nt, Artificial Sequence
  Description of Artificial Sequence Synthetic oligonucleotide
cauccuugggauuacgcucccucaa

SEQ ID NO: 181 - RNA, 25 nt, Artificial Sequence
  Description of Artificial Sequence Synthetic oligonucleotide
uugagggagcguaaucccaaggaug

SEQ ID NO: 182 - RNA, 25 nt, Artificial Sequence
  Description of Artificial Sequence Synthetic oligonucleotide
cccucaaggagauaagugauggaga

SEQ ID NO: 183 - RNA, 25 nt, Artificial Sequence
  Description of Artificial Sequence Synthetic oligonucleotide
ucuccaucacuuaucuccuugaggg

SEQ ID NO: 184 - RNA, 25 nt, Artificial Sequence
  Description of Artificial Sequence Synthetic oligonucleotide
ccucaaggagauaagugauggagau

SEQ ID NO: 185 - RNA, 25 nt, Artificial Sequence
  Description of Artificial Sequence Synthetic oligonucleotide
aucuccaucacuuaucuccuugagg

SEQ ID NO: 186 - RNA, 25 nt, Artificial Sequence
  Description of Artificial Sequence Synthetic oligonucleotide
ucaaggagauaagugauggagaugu

SEQ ID NO: 187 - RNA, 25 nt, Artificial Sequence
  Description of Artificial Sequence Synthetic oligonucleotide
acaucuccaucacuuaucuccuuga

SEQ ID NO: 188 - RNA, 25 nt, Artificial Sequence
  Description of Artificial Sequence Synthetic oligonucleotide
aggagauaagugauggagaugugau

SEQ ID NO: 189 - RNA, 25 nt, Artificial Sequence
  Description of Artificial Sequence Synthetic oligonucleotide
aucacaucuccaucacuuaucuccu

SEQ ID NO: 190 - RNA, 25 nt, Artificial Sequence
  Description of Artificial Sequence Synthetic oligonucleotide
ggagauaagugauggagaugugaua

SEQ ID NO: 191 - RNA, 25 nt, Artificial Sequence
  Description of Artificial Sequence Synthetic oligonucleotide
uaucacaucuccaucacuuaucucc

SEQ ID NO: 192 - RNA, 25 nt, Artificial Sequence
  Description of Artificial Sequence Synthetic oligonucleotide
gagauaagugauggagaugugauaa

SEQ ID NO: 193 - RNA, 25 nt, Artificial Sequence
  Description of Artificial Sequence Synthetic oligonucleotide
uuaucacaucuccaucacuuaucuc

SEQ ID NO: 194 - RNA, 25 nt, Artificial Sequence
  Description of Artificial Sequence Synthetic oligonucleotide
ccaagggaguuuguggagaacucug

SEQ ID NO: 195 - RNA, 25 nt, Artificial Sequence
  Description of Artificial Sequence Synthetic oligonucleotide
cagaguucuccacaaacucccuugg

SEQ ID NO: 196 - RNA, 25 nt, Artificial Sequence
  Description of Artificial Sequence Synthetic oligonucleotide
gaguuuguggagaacucugagugca

SEQ ID NO: 197 - RNA, 25 nt, Artificial Sequence
  Description of Artificial Sequence Synthetic oligonucleotide
ugcacucagaguucuccacaaacuc

SEQ ID NO: 198 - RNA, 25 nt, Artificial Sequence
  Description of Artificial Sequence Synthetic oligonucleotide
cagacaacuguauccagugugccca

SEQ ID NO: 199 - RNA, 25 nt, Artificial Sequence
  Description of Artificial Sequence Synthetic oligonucleotide
ugggcacacuggauacaguugucug

SEQ ID NO: 200 - RNA, 25 nt, Artificial Sequence
  Description of Artificial Sequence Synthetic oligonucleotide
uguauccagugugcccacuacauug

SEQ ID NO: 201 - RNA, 25 nt, Artificial Sequence
  Description of Artificial Sequence Synthetic oligonucleotide
caauguagugggcacacuggauaca

SEQ ID NO: 202 - RNA, 25 nt, Artificial Sequence
  Description of Artificial Sequence Synthetic oligonucleotide
cauccaaacugcaccuacggaugca

SEQ ID NO: 203 - RNA, 25 nt, Artificial Sequence
  Description of Artificial Sequence Synthetic oligonucleotide
ugcauccguaggugcaguuuggaug

SEQ ID NO: 204 - RNA, 25 nt, Artificial Sequence
  Description of Artificial Sequence Synthetic oligonucleotide
gcacgguguauaagggacucuggau

SEQ ID NO: 205 - RNA, 25 nt, Artificial Sequence
  Description of Artificial Sequence Synthetic oligonucleotide
auccagagucccuuauacaccgugc

SEQ ID NO: 206 - RNA, 25 nt, Artificial Sequence
  Description of Artificial Sequence Synthetic oligonucleotide
ccgucgcuaucaaggaauuaagaga

SEQ ID NO: 207 - RNA, 25 nt, Artificial Sequence
  Description of Artificial Sequence Synthetic oligonucleotide
ucucuuaauuccuugauagcgacgg

SEQ ID NO: 208 - RNA, 25 nt, Artificial Sequence
  Description of Artificial Sequence Synthetic oligonucleotide
cgucgcuaucaaggaauuaagagaa

SEQ ID NO: 209 - RNA, 25 nt, Artificial Sequence
  Description of Artificial Sequence Synthetic oligonucleotide
uucucuuaauuccuugauagcgacg

SEQ ID NO: 210 - RNA, 25 nt, Artificial Sequence
  Description of Artificial Sequence Synthetic oligonucleotide
cgcuaucaaggaauuaagagaagca

SEQ ID NO: 211 - RNA, 25 nt, Artificial Sequence
  Description of Artificial Sequence Synthetic oligonucleotide
ugcuucucuuaauuccuugauagcg

SEQ ID NO: 212 - RNA, 25 nt, Artificial Sequence
  Description of Artificial Sequence Synthetic oligonucleotide
caaggaauuaagagaagcaacaucu

SEQ ID NO: 213 - RNA, 25 nt, Artificial Sequence
  Description of Artificial Sequence Synthetic oligonucleotide
agauguugcuucucuuaauuccuug

SEQ ID NO: 214 - RNA, 25 nt, Artificial Sequence
  Description of Artificial Sequence Synthetic oligonucleotide
gaaauccucgaugaagccuacguga

SEQ ID NO: 215 - RNA, 25 nt, Artificial Sequence
  Description of Artificial Sequence Synthetic oligonucleotide
ucacguaggcuucaucgaggauuuc

SEQ ID NO: 216 - RNA, 25 nt, Artificial Sequence
  Description of Artificial Sequence Synthetic oligonucleotide
ccgcagcaugucaagaucacagauu

SEQ ID NO: 217 - RNA, 25 nt, Artificial Sequence
  Description of Artificial Sequence Synthetic oligonucleotide
aaucugugaucuugacaugcugcgg

SEQ ID NO: 218 - RNA, 25 nt, Artificial Sequence
  Description of Artificial Sequence Synthetic oligonucleotide
cgcagcaugucaagaucacagauuu

SEQ ID NO: 219 - RNA, 25 nt, Artificial Sequence
  Description of Artificial Sequence Synthetic oligonucleotide
aaaucugugaucuugacaugcugcg

SEQ ID NO: 220 - RNA, 25 nt, Artificial Sequence
  Description of Artificial Sequence Synthetic oligonucleotide
caugcagaaggaggcaaagugccua

SEQ ID NO: 221 - RNA, 25 nt, Artificial Sequence
  Description of Artificial Sequence Synthetic oligonucleotide
uaggcacuuugccuccuucugcaug

SEQ ID NO: 222 - RNA, 25 nt, Artificial Sequence
  Description of Artificial Sequence Synthetic oligonucleotide
cagaaggaggcaaagugccuaucaa

SEQ ID NO: 223 - RNA, 25 nt, Artificial Sequence
  Description of Artificial Sequence Synthetic oligonucleotide
uugauaggcacuuugccuccuucug

SEQ ID NO: 224 - RNA, 25 nt, Artificial Sequence
  Description of Artificial Sequence Synthetic oligonucleotide
aagugccuaucaaguggauggcauu

SEQ ID NO: 225 - RNA, 25 nt, Artificial Sequence
  Description of Artificial Sequence Synthetic oligonucleotide
aaugccauccacuugauaggcacuu

SEQ ID NO: 226 - RNA, 25 nt, Artificial Sequence
  Description of Artificial Sequence Synthetic oligonucleotide
gccuaucaaguggauggcauuggaa

SEQ ID NO: 227 - RNA, 25 nt, Artificial Sequence
  Description of Artificial Sequence Synthetic oligonucleotide
uuccaaugccauccacuugauaggc

SEQ ID NO: 228 - RNA, 25 nt, Artificial Sequence
  Description of Artificial Sequence Synthetic oligonucleotide
ccuaucaaguggauggcauuggaau

SEQ ID NO: 229 - RNA, 25 nt, Artificial Sequence
  Description of Artificial Sequence Synthetic oligonucleotide
auuccaaugccauccacuugauagg

SEQ ID NO: 230 - RNA, 25 nt, Artificial Sequence
  Description of Artificial Sequence Synthetic oligonucleotide
caaguggauggcauuggaaucaauu

SEQ ID NO: 231 - RNA, 25 nt, Artificial Sequence
  Description of Artificial Sequence Synthetic oligonucleotide
aauugauuccaaugccauccacuug

SEQ ID NO: 232 - RNA, 25 nt, Artificial Sequence
  Description of Artificial Sequence Synthetic oligonucleotide
cagccacccauauguaccaucgaug

SEQ ID NO: 233 - RNA, 25 nt, Artificial Sequence
  Description of Artificial Sequence Synthetic oligonucleotide
caucgaugguacauauggguggcug

SEQ ID NO: 234 - RNA, 25 nt, Artificial Sequence
  Description of Artificial Sequence Synthetic oligonucleotide
cacccauauguaccaucgaugucua

SEQ ID NO: 235 - RNA, 25 nt, Artificial Sequence
  Description of Artificial Sequence Synthetic oligonucleotide
uagacaucgaugguacauaugggug

SEQ ID NO: 236 - RNA, 25 nt, Artificial Sequence
  Description of Artificial Sequence Synthetic oligonucleotide
uguaccaucgaugucuacaugauca

SEQ ID NO: 237 - RNA, 25 nt, Artificial Sequence
  Description of Artificial Sequence Synthetic oligonucleotide
ugaucauguagacaucgaugguaca

SEQ ID NO: 238 - RNA, 25 nt, Artificial Sequence
  Description of Artificial Sequence Synthetic oligonucleotide
uagacgcagauagucgcccaaaguu

SEQ ID NO: 239 - RNA, 25 nt, Artificial Sequence
  Description of Artificial Sequence Synthetic oligonucleotide
aacuuugggcgacuaucugcgucua

SEQ ID NO: 240 - RNA, 25 nt, Artificial Sequence
  Description of Artificial Sequence Synthetic oligonucleotide
ccaaguccuacagacuccaacuucu

SEQ ID NO: 241 - RNA, 25 nt, Artificial Sequence
  Description of Artificial Sequence Synthetic oligonucleotide
agaaguuggagucuguaggacuugg

SEQ ID NO: 242 - RNA, 25 nt, Artificial Sequence
  Description of Artificial Sequence Synthetic oligonucleotide
caaguccuacagacuccaacuucua

SEQ ID NO: 243 - RNA, 25 nt, Artificial Sequence
  Description of Artificial Sequence Synthetic oligonucleotide
uagaaguuggagucuguaggacuug

SEQ ID NO: 244 - RNA, 25 nt, Artificial Sequence
  Description of Artificial Sequence Synthetic oligonucleotide
acuccaacuucuaccgugcccugau

SEQ ID NO: 245 - RNA, 25 nt, Artificial Sequence
  Description of Artificial Sequence Synthetic oligonucleotide
aucagggcacgguagaaguuggagu

SEQ ID NO: 246 - RNA, 25 nt, Artificial Sequence
  Description of Artificial Sequence Synthetic oligonucleotide
ucucugagugcaaccagcaacaauu

SEQ ID NO: 247 - RNA, 25 nt, Artificial Sequence
  Description of Artificial Sequence Synthetic oligonucleotide
aauuguugcugguugcacucagaga

SEQ ID NO: 248 - RNA, 25 nt, Artificial Sequence
  Description of Artificial Sequence Synthetic oligonucleotide
caauuccaccguggcuugcauugau

SEQ ID NO: 249 - RNA, 25 nt, Artificial Sequence
  Description of Artificial Sequence Synthetic oligonucleotide
aucaaugcaagccacgguggaauug

SEQ ID NO: 250 - RNA, 25 nt, Artificial Sequence
  Description of Artificial Sequence Synthetic oligonucleotide
ccaccguggcuugcauugauagaaa

SEQ ID NO: 251 - RNA, 25 nt, Artificial Sequence
  Description of Artificial Sequence Synthetic oligonucleotide
uuucuaucaaugcaagccacggugg

SEQ ID NO: 252 - RNA, 25 nt, Artificial Sequence
  Description of Artificial Sequence Synthetic oligonucleotide
caccguggcuugcauugauagaaau

SEQ ID NO: 253 - RNA, 25 nt, Artificial Sequence
  Description of Artificial Sequence Synthetic oligonucleotide
auuucuaucaaugcaagccacggug

SEQ ID NO: 254 - RNA, 25 nt, Artificial Sequence
  Description of Artificial Sequence Synthetic oligonucleotide
ugcauugauagaaaugggcugcaaa

SEQ ID NO: 255 - RNA, 25 nt, Artificial Sequence
  Description of Artificial Sequence Synthetic oligonucleotide
uuugcagcccauuucuaucaaugca

SEQ ID NO: 256 - RNA, 25 nt, Artificial Sequence
  Description of Artificial Sequence Synthetic oligonucleotide
gcauugauagaaaugggcugcaaag

SEQ ID NO: 257 - RNA, 25 nt, Artificial Sequence
  Description of Artificial Sequence Synthetic oligonucleotide
cuuugcagcccauuucuaucaaugc

SEQ ID NO: 258 - RNA, 25 nt, Artificial Sequence
  Description of Artificial Sequence Synthetic oligonucleotide
cagcuucuugcagcgauacagcuca

SEQ ID NO: 259 - RNA, 25 nt, Artificial Sequence
  Description of Artificial Sequence Synthetic oligonucleotide
ugagcuguaucgcugcaagaagcug

SEQ ID NO: 260 - RNA, 25 nt, Artificial Sequence
  Description of Artificial Sequence Synthetic oligonucleotide
ccagugccugaauacauaaaccagu

SEQ ID NO: 261 - RNA, 25 nt, Artificial Sequence
  Description of Artificial Sequence Synthetic oligonucleotide
acugguuuauguauucaggcacugg

SEQ ID NO: 262 - RNA, 25 nt, Artificial Sequence
  Description of Artificial Sequence Synthetic oligonucleotide
ccaccugugucaacagcacauucga

SEQ ID NO: 263 - RNA, 25 nt, Artificial Sequence
  Description of Artificial Sequence Synthetic oligonucleotide
ucgaaugugcuguugacacaggugg

SEQ ID NO: 264 - RNA, 25 nt, Artificial Sequence
  Description of Artificial Sequence Synthetic oligonucleotide
gcccagaaaggcagccaccaaauua

SEQ ID NO: 265 - RNA, 25 nt, Artificial Sequence
  Description of Artificial Sequence Synthetic oligonucleotide
uaauuugguggcugccuuucugggc

SEQ ID NO: 266 - RNA, 25 nt, Artificial Sequence
  Description of Artificial Sequence Synthetic oligonucleotide
cccagaaaggcagccaccaaauuag

SEQ ID NO: 267 - RNA, 25 nt, Artificial Sequence
  Description of Artificial Sequence Synthetic oligonucleotide
cuaauuugguggcugccuuucuggg

SEQ ID NO: 268 - RNA, 25 nt, Artificial Sequence
  Description of Artificial Sequence Synthetic oligonucleotide
ggaagccaagccaaauggcaucuuu

SEQ ID NO: 269 - RNA, 25 nt, Artificial Sequence
  Description of Artificial Sequence Synthetic oligonucleotide
aaagaugccauuuggcuuggcuucc

SEQ ID NO: 270 - RNA, 25 nt, Artificial Sequence
  Description of Artificial Sequence Synthetic oligonucleotide
gaagccaagccaaauggcaucuuua

SEQ ID NO: 271 - RNA, 25 nt, Artificial Sequence
  Description of Artificial Sequence Synthetic oligonucleotide
uaaagaugccauuuggcuuggcuuc

SEQ ID NO: 272 - RNA, 25 nt, Artificial Sequence
  Description of Artificial Sequence Synthetic oligonucleotide
aagccaagccaaauggcaucuuuaa

SEQ ID NO: 273 - RNA, 25 nt, Artificial Sequence
  Description of Artificial Sequence Synthetic oligonucleotide
uuaaagaugccauuuggcuuggcuu

SEQ ID NO: 274 - RNA, 25 nt, Artificial Sequence
  Description of Artificial Sequence Synthetic oligonucleotide
uggcaucuuuaagggcuccacagcu

SEQ ID NO: 275 - RNA, 25 nt, Artificial Sequence
  Description of Artificial Sequence Synthetic oligonucleotide
agcuguggagcccuuaaagaugcca

SEQ ID NO: 276 - RNA, 25 nt, Artificial Sequence
  Description of Artificial Sequence Synthetic oligonucleotide
caucuuuaagggcuccacagcugaa

SEQ ID NO: 277 - RNA, 25 nt, Artificial Sequence
  Description of Artificial Sequence Synthetic oligonucleotide
uucagcuguggagcccuuaaagaug

SEQ ID NO: 278 - RNA, 25 nt, Artificial Sequence
  Description of Artificial Sequence Synthetic oligonucleotide
ccacggaggauaguaugagcccuaa

SEQ ID NO: 279 - RNA, 25 nt, Artificial Sequence
  Description of Artificial Sequence Synthetic oligonucleotide
uuagggcucauacuauccuccgugg

SEQ ID NO: 280 - RNA, 25 nt, Artificial Sequence
  Description of Artificial Sequence Synthetic oligonucleotide
cacggaggauaguaugagcccuaaa

SEQ ID NO: 281 - RNA, 25 nt, Artificial Sequence
  Description of Artificial Sequence Synthetic oligonucleotide
uuuagggcucauacuauccuccgug

SEQ ID NO: 282 - RNA, 25 nt, Artificial Sequence
  Description of Artificial Sequence Synthetic oligonucleotide
uacagaaacgcauccagcaagaaua

SEQ ID NO: 283 - RNA, 25 nt, Artificial Sequence
  Description of Artificial Sequence Synthetic oligonucleotide
uauucuugcuggaugcguuucugua

SEQ ID NO: 284 - RNA, 25 nt, Artificial Sequence
  Description of Artificial Sequence Synthetic oligonucleotide
ugauggaccagugguuuccagucau

SEQ ID NO: 285 - RNA, 25 nt, Artificial Sequence
  Description of Artificial Sequence Synthetic oligonucleotide
augacuggaaaccacugguccauca

SEQ ID NO: 286 - RNA, 25 nt, Artificial Sequence
  Description of Artificial Sequence Synthetic oligonucleotide
cagugguuuccagucaugagcguua

SEQ ID NO: 287 - RNA, 25 nt, Artificial Sequence
  Description of Artificial Sequence Synthetic oligonucleotide
uaacgcucaugacuggaaaccacug

SEQ ID NO: 288 - RNA, 25 nt, Artificial Sequence
  Description of Artificial Sequence Synthetic oligonucleotide
cagcaagagaggaugacacaucaaa

SEQ ID NO: 289 - RNA, 25 nt, Artificial Sequence
  Description of Artificial Sequence Synthetic oligonucleotide
uuugaugugucauccucucuugcug

SEQ ID NO: 290 - RNA, 25 nt, Artificial Sequence
  Description of Artificial Sequence Synthetic oligonucleotide
ccagcccacauuggauucaucagca

SEQ ID NO: 291 - RNA, 25 nt, Artificial Sequence
  Description of Artificial Sequence Synthetic oligonucleotide
ugcugaugaauccaaugugggcugg

SEQ ID NO: 292 - RNA, 25 nt, Artificial Sequence
  Description of Artificial Sequence Synthetic oligonucleotide
cagcccacauuggauucaucagcau

SEQ ID NO: 293 - RNA, 25 nt, Artificial Sequence
  Description of Artificial Sequence Synthetic oligonucleotide
augcugaugaauccaaugugggcug

SEQ ID NO: 294 - RNA, 25 nt, Artificial Sequence
  Description of Artificial Sequence Synthetic oligonucleotide
gcccacauuggauucaucagcauuu

SEQ ID NO: 295 - RNA, 25 nt, Artificial Sequence
  Description of Artificial Sequence Synthetic oligonucleotide
aaaugcugaugaauccaaugugggc

SEQ ID NO: 296 - RNA, 25 nt, Artificial Sequence
  Description of Artificial Sequence Synthetic oligonucleotide
ccacagcugagaauguggaauaccu

SEQ ID NO: 297 - RNA, 25 nt, Artificial Sequence
  Description of Artificial Sequence Synthetic oligonucleotide
agguauuccacauucucagcugugg

SEQ ID NO: 298 - RNA, 25 nt, Artificial Sequence
  Description of Artificial Sequence Synthetic oligonucleotide
cacagcugagaauguggaauaccua

SEQ ID NO: 299 - RNA, 25 nt, Artificial Sequence
  Description of Artificial Sequence Synthetic oligonucleotide
uagguauuccacauucucagcugug

SEQ ID NO: 300 - RNA, 25 nt, Artificial Sequence
  Description of Artificial Sequence Synthetic oligonucleotide
ucuccuaauuugaggcucagaugaa

SEQ ID NO: 301 - RNA, 25 nt, Artificial Sequence
  Description of Artificial Sequence Synthetic oligonucleotide
uucaucugagccucaaauuaggaga

SEQ ID NO: 302 - RNA, 25 nt, Artificial Sequence
  Description of Artificial Sequence Synthetic oligonucleotide
gaggcucagaugaaaugcaucaggu

SEQ ID NO: 303 - RNA, 25 nt, Artificial Sequence
  Description of Artificial Sequence Synthetic oligonucleotide
accugaugcauuucaucugagccuc

SEQ ID NO: 304 - RNA, 25 nt, Artificial Sequence
  Description of Artificial Sequence Synthetic oligonucleotide
caggugcgaaugacaguagcauuau

SEQ ID NO: 305 - RNA, 25 nt, Artificial Sequence
  Description of Artificial Sequence Synthetic oligonucleotide
auaaugcuacugucauucgcaccug

SEQ ID NO: 306 - RNA, 25 nt, Artificial Sequence
  Description of Artificial Sequence Synthetic oligonucleotide
gcgaaugacaguagcauuaugagua

SEQ ID NO: 307 - RNA, 25 nt, Artificial Sequence
  Description of Artificial Sequence Synthetic oligonucleotide
uacucauaaugcuacugucauucgc

SEQ ID NO: 308 - RNA, 25 nt, Artificial Sequence
  Description of Artificial Sequence Synthetic oligonucleotide
caguagcauuaugaguaguguggaa

SEQ ID NO: 309 - RNA, 25 nt, Artificial Sequence
  Description of Artificial Sequence Synthetic oligonucleotide
uuccacacuacucauaaugcuacug

SEQ ID NO: 310 - RNA, 25 nt, Artificial Sequence
  Description of Artificial Sequence Synthetic oligonucleotide
gcauuaugaguaguguggaauucag

SEQ ID NO: 311 - RNA, 25 nt, Artificial Sequence
  Description of Artificial Sequence Synthetic oligonucleotide
cugaauuccacacuacucauaaugc

SEQ ID NO: 312 - RNA, 25 nt, Artificial Sequence
  Description of Artificial Sequence Synthetic oligonucleotide
aguaguguggaauucagguaguaaa

SEQ ID NO: 313 - RNA, 25 nt, Artificial Sequence
  Description of Artificial Sequence Synthetic oligonucleotide
uuuacuaccugaauuccacacuacu

SEQ ID NO: 314 - RNA, 25 nt, Artificial Sequence
  Description of Artificial Sequence Synthetic oligonucleotide
ugugcccuguaaccugacugguuaa

SEQ ID NO: 315 - RNA, 25 nt, Artificial Sequence
  Description of Artificial Sequence Synthetic oligonucleotide
uuaaccagucagguuacagggcaca

SEQ ID NO: 316 - RNA, 25 nt, Artificial Sequence
  Description of Artificial Sequence Synthetic oligonucleotide
ccugacugguuaacagcaguccuuu

SEQ ID NO: 317 - RNA, 25 nt, Artificial Sequence
  Description of Artificial Sequence Synthetic oligonucleotide
aaaggacugcuguuaaccagucagg

SEQ ID NO: 318 - RNA, 25 nt, Artificial Sequence
  Description of Artificial Sequence Synthetic oligonucleotide
gacugguuaacagcaguccuuugua

SEQ ID NO: 319 - RNA, 25 nt, Artificial Sequence
  Description of Artificial Sequence Synthetic oligonucleotide
uacaaaggacugcuguuaaccaguc

SEQ ID NO: 320 - RNA, 25 nt, Artificial Sequence
  Description of Artificial Sequence Synthetic oligonucleotide
cagcaguccuuuguaaacaguguuu

SEQ ID NO: 321 - RNA, 25 nt, Artificial Sequence
  Description of Artificial Sequence Synthetic oligonucleotide
aaacacuguuuacaaaggacugcug

SEQ ID NO: 322 - RNA, 25 nt, Artificial Sequence
  Description of Artificial Sequence Synthetic oligonucleotide
cagccuacaguuauguucagucaca

SEQ ID NO: 323 - RNA, 25 nt, Artificial Sequence
  Description of Artificial Sequence Synthetic oligonucleotide
ugugacugaacauaacuguaggcug

SEQ ID NO: 324 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 325 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 326 - protein, 1210 aa, Homo sapiens [sequence omitted]

SEQ ID NO: 327 - RNA, 25 nt, Artificial Sequence
  Description of Artificial Sequence Synthetic oligonucleotide
ggaaggcccauugaguccaacuaca

SEQ ID NO: 328 - RNA, 25 nt, Artificial Sequence
  Description of Artificial Sequence Synthetic oligonucleotide
uguaguuggacucaaugggccuucc

SEQ ID NO: 329 - protein, 17 aa, Artificial Sequence
  Description of Artificial Sequence Synthetic peptide
KHHHKHHHKHHHKHHHK

(19) United States
(12) Patent Application Publication
(10) Pub. No.: US 2011/0046067 A1
(43) Pub. Date: Feb. 24, 2011

(54) COMPOSITIONS COMPRISING HUMAN EGFR-siRNA AND METHODS OF USE

(75) Inventors: Xiaodong Yang, Palo Alto, CA (US); Frank Y. Xie, Germantown, MD (US); Yijia Liu, Gaithersburg, MD (US); Ying Liu, Palo Alto, CA (US)

Correspondence Address: ROPES & GRAY LLP, PATENT DOCKETING 39/361, 1211 AVENUE OF THE AMERICAS, NEW YORK, NY, 10036-8704, US

(73) Assignee: Intradigm Corporation, Palo Alto, CA (US)

(21) Appl. No.: 12/665,819

(22) PCT Filed: Jun. 20, 2008
(86) PCT No.: PCT/US08/07672

§ 371 Date: Feb. 1, 2010

Related U.S. Application Data
(60) Provisional application No. 61/060,721, filed on Jun. 11, 2008.
(60) Provisional application No. 61/124,223, filed on Apr. 14, 2008.
(60) Provisional application No. 60/998,284, filed on Oct. 10, 2007.
(60) Provisional application No. 60/945,842, filed on Jun. 22, 2007.

(51) Int. Cl. A61K 38/02; C07H 21/02; A61K 31/713; C12N 5/071; A61P 35/00

(52) U.S. Cl. 514/19.3; 536/24.5; 514/44 A; 435/375

(57) ABSTRACT

The present invention provides nucleic acid molecules that inhibit EGFR expression. Methods of using the nucleic acid molecules are also provided.

### CROSS-REFERENCE TO RELATED APPLICATIONS

This application claims priority under 35 U.S.C. §119(e) from U.S. provisional application 60/945,842, filed Jun. 22, 2007, U.S. provisional application 60/998,284, filed Oct. 10, 2007, U.S. provisional application 61/124,223, filed Apr. 14, 2008 and U.S. provisional application 61/060,721, filed Jun. 11, 2008.

### FIELD OF THE INVENTION

The present invention is in the field of molecular biology and medicine and relates to short interfering RNA (siRNA) molecules for modulating the expression of Epidermal Growth Factor (EGF) receptor.

### BACKGROUND OF THE INVENTION

EGFR, is a 170 kDa transmembrane glycoprotein that has been shown to play an important role in controlling cell proliferation and differentiation. EGFR is a member of the ErbB family of receptors, that includes EGFR (ErbB-1), HER2/c-neu (ErbB-2), Her 3 (ErbB-3) and Her 4 (ErbB-4). EGFR is composed of extracellular, transmembrane and cytoplasmic domains. Ligand binding to the extacellular domain of EGFR leads to dimerization and activation of a tyrosine kinase activity, initiating a complex cascade of enzymatic and biological events leading to cell proliferation and differentiation.

Overexpression of EGFR has been associated with many malignancies, including ones of the lung, kidney, pancreas, breast, head and neck, stomach and colon. Various cells have been shown to produce variant form(s) of EGFR. A431 human epidermoid carcinoma cells, for example, have been shown to produce a truncated EGFR.

The role of EGFR in cancer has been validated by the recent FDA approval of several EGFR inhibitors including neutralizing antibodies such as Vectibix and Erbitux and small molecule TKi such as Tarceva™ for the treatment of metastatic colon, lung, pancreas, and head and neck cancers.

RNA interference (RNAi) technology is emerging as an effective means for reducing the expression of specific gene products and may therefore prove to be uniquely useful in a number of therapeutic, diagnostic, and research applications for the modulation of expression of EGFR. The present invention provides compositions and methods for modulating expression of these proteins using RNAi technology.

Thus, there is a need in the art for compositions and methods for modulating the expression of EGFR as a therapeutic approach for the treatment of cancer and other diseases. The present invention provides this and other advantages.

### SUMMARY OF THE INVENTION

One aspect of the present invention provides a nucleic acid molecule that down regulates expression of an epidermal growth factor (EGF) receptor gene, wherein the nucleic acid molecule comprises a nucleotide sequence that targets EGFR mRNA, wherein the nucleic acid molecule comprises a nucleotide sequence that targets any one of the polynucleotide sequences set forth in SEQ ID NOs: 1-10 or 21-121. In a particular embodiment, the nucleic acid is an siRNA molecule. In more particular embodiments, the siRNA comprises any one of the single stranded RNA sequences provided in SEQ ID NOs: 11-20 and 122-323, or a double-stranded RNA thereof. In certain embodiments, the nucleic acid molecule down regulates expression of an EGFR gene via RNA interference (RNAi).

In another embodiment, the present invention provides for a composition comprising any one or more of the siRNA molecules, wherein the siRNA comprises any one of the single stranded RNA sequences provided in SEQ ID NOs: 11-20 and 122-323, or a double-stranded RNA thereof. In this regard, the composition may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more siRNA molecules of the invention. In particular embodiments, the siRNA comprises a targeting moiety.

In various embodiments, the present invention provides a method for treating or preventing a cancer in a subject with an EGFR expressing cancer and having or suspected of being at risk for having the cancer, comprising administering to a subject a composition comprising any one of the single stranded RNA sequences provided in SEQ ID NOs: 11-20 and 122-323, or a double-stranded RNA thereof, thereby treating or preventing the cancer. In certain embodiments, the cancer is selected from the group consisting of breast cancer, lung cancer, prostate cancer, colorectal cancer, brain cancer, esophageal cancer, stomach cancer, bladder cancer, pancreatic cancer, cervical cancer, head and neck cancer, kidney cancer, endometrial cancer, ovarian cancer, meningioma, melanoma, lymphoma, and glioblastoma.

In another embodiment, the present invention provides a method for inhibiting the synthesis or expression of EGFR comprising contacting a cell expressing EGFR with one or more siRNAs, wherein the siRNAs comprise a sequence as set forth in SEQ ID NOs: 11-20 or 122-323.

These and other aspects of the present invention will become apparent upon references to the following detailed description.

### BRIEF DESCRIPTION OF THE DRAWING(S)

FIG. 1 is a bar graph depicting in vitro inhibition of hEGFR by certain siRNA molecules.

Human EGFR gene silencing activity of human EGFR-siRNA was tested in HT-29 cells. The HT-29 cells were transfected with the EGFR-siRNAs using an Electroporation mediated transfection method with 4 or 8 ug siRNA per 2×106 cells/200 ul. The concentration of EGFR protein in the transfected HT-29 cells was measured at 72 hours post transfection using a hEGFR ELISA kit (R&D Systems, Inc.). The concentration of hEGFR protein was normalized against total cellular protein and the percentage of hEGFR inhibition was normalized against cells treated with a mock process without siRNA. All 5 hEGFR siRNA demonstrated inhibition of hEGFR production. The hEGFR-25-1 and hEGFR-25-2 were the most potent siRNA with a more than 70% inhibition of hEGFR protein at 72 hours post siRNA transfection.

FIG. 2 is a bar graph demonstrating the inhibition of hEGFR by two siRNA molecules in a dose-dependent manner.

The selected hEGFR-siRNA, hEGFR-25-1 and hEGFR-25-2, were tested for their capability of inhibiting hEGFR expression in a dose-dependent manner. The HT-29 cells were transfected with the EGFR-siRNAs in a range of 0.01-10 ug siRNA per 2×10⁶ cells/200 μl using an electroporation mediated transfection method. The concentration of EGFR protein in the transfected HT-29 cells was measured at 48 hours post transfection using a hEGFR ELISA kit (R&D Systems, Inc.). The concentration of hEGFR protein was normalized against total cellular protein and the percentage of hEGFR inhibition was normalized against cells treated with a mock process without siRNA. Both hEGFR-25-1 and hEGFR-25-2 demonstrated a dose-dependent inhibition of hEGFR production.

FIG. 3 is a line graph demonstrating the tumor inhibition effect of hEGFR-siRNA-PolyTran™ NPX on A431 tumor xenografts.

Antitumor efficacy of PolyTran™ (PT-NPX) carrying hEGFR-siRNA was determined in A431 (epidermoid carcinoma) xenograft model. Mice bearing established A431 tumors were treated with intravenous administration of PolyTran NPX carrying hEGFR-siRNA every other day for 6 times started on Day 4 post tumor cells implantation. Treatment controls included no treatment (untreated) and Erlotinib (Tarceva™) which was daily administered orally at 100 mg/kg for 6 days. Treatment with PT-NPX carrying human EGFR siRNA significantly inhibited A431 tumor growth in comparison with untreated control; and the inhibition effect was more profound than the Tarceva™ treatment control.

FIG. 4 is a line graph demonstrating the inhibition of A431 tumor growth by PT-EGFR-siRNA NPX is hEGFR-siRNA specific and requires formulation of PT-siRNA NPX.

PolyTran™ nanoparticles (PT-NPX) carrying hEGFR-siRNA or negative control-siRNA, as well as the PolyTran peptide alone or hEGFR-siRNA alone, were tested in A431 (epidermoid carcinoma) xenograft model. Mice bearing established A431 tumors were treated with intravenous administration of PolyTran NPX carrying hEGFR-siRNA or negative control-siRNA (2 mg/kg), or hEGFR-siRNA alone, or PolyTran peptide alone every other day for 4 times started on Day 5 post tumor cells implantation. Treatment controls included no treatment (untreated). Only the treatment with PT-NPX carrying human EGFR siRNA significantly inhibited A431 tumor growth in comparison with untreated control. All other treatment groups include PT-NPX carrying control-siRNA, hEGFR-siRNA alone, or PolyTran peptide, did not inhibit A431 tumor growth.

FIG. 5 is a line graph demonstrating the tumor inhibition effect of hEGFR-siRNA-PolyTran™ NPX on A549 tumor xenografts.

The antitumor efficacy of PolyTran™ (PT-NPX) carrying hEGFR-siRNA was determined in A549 (NSCLC) xenograft model. Mice bearing established A549 tumors were treated with intravenous administration of PolyTran NPX carrying hEGFR-siRNA every other day for 6 times started on Day 9 post tumor cells implantation. Treatment controls included no treatment (untreated) and Erlotinib (Tarceva™) which was daily administered orally at 100 mg/kg for 6 days. Treatment with PT-NPX carrying human EGFR siRNA significantly inhibited A549 tumor growth in comparison with untreated control; and the inhibition effect was more profound than the Tarceva™ treatment control. The PT-NPX carrying control-siRNA did not have inhibition effect on A549 tumor growth.

FIG. 6 is a schematic showing the structure and composition of the PolyTran™. PolyTran™ is a synthetic biodegradable cationic branched polypeptide. The positively charged PolyTran™ polypeptide serves as a carrier and condenser for the negatively charged siRNA.

FIG. 7 is a diagram showing the histidine-lysine H3K4b polypeptide structure. The histidine-lysine H3K4b polypeptide was used in the formulation of PT-NPX.

FIG. 8 is an electronic image of PolyTran-siRNA NPX. When PolyTran polypeptide was mixed with siRNA (against hVEGF), spherical shaped nanoparticles (PT-siRNA NPX) with diameter around 100 nm were formed in solution.

FIG. 9 shows fluorescent microscope images demonstrating cellular uptake of PT-siRNA NPX. Mouse endothelial EA.hy926 cells were transfected with the PT-NPX containing Alexa488-labeled hVEGF siRNA (QIAGEN) at equivalent siRNA concentration of 5 ug/mL for 6 hours. The fluorescence observed within the cells suggest internalization of the PT-siRNA NPX.

FIG. 10 shows fluorescent microscope images demonstrating tissue distribution of PT-siRNA NPX in tumors. Biodistribution of the PT-NPX following i.v. injection was investigated using the PT NPX carrying fluorescently labeled siRNA (Alexa-555 labeled hVEGF siRNA from QIAGEN). Nude mice bearing A431 xenografts were injected intravenously. with the PT-NPX. One hour post injection, the tumor tissues were removed and frozen tissue sections were prepared. Fluorescence labeled siRNA was found in the tumor tissue, indicating distribution of the PT-NPX in tumor tissue was achieved. No auto-fluorescent background is seen in the untreated tumor tissues.

FIGS. 11A-C are line graphs demonstrating hVEGF gene silencing by VEGF siRNA. Target gene silencing activity of human VEGF-siRNA was tested in human prostate cancer PC-3 cells. The cells were transfected with the siRNA using LipoFectamine RNAiMax (Invitrogen). The concentration of VEGF in the media were measured at 24 (FIG. 11A), 48 (FIG. 11B) and 72 (FIG. 11C) hours post transfection using an ELISA kit (R&D Systems, Inc.). All three human VEGF siRNA tested inhibited the production of VEGF in a dose-dependent manner and have nano- or subnano-molar potency.

FIG. 12 is a bar graph demonstrating hVEGF gene silencing by PT-siRNA NPX. The human prostate cancer cell line PC-3, which expresses VEGF, was treated with PT-NPX carrying hVEGF-siRNA or Control siRNA in serum-free medium for 4 hours, and then replenished with serum (10%). 72 hours after the treatment, cell lysates were collected for the measurement of VEGF using an ELISA kit (R&D Systems, Inc.). PT-NPX containing hVEGF siRNA suppressed hVEGF production in vitro.

FIG. 13 is a line graph showing the effect of PT-siRNA NPX on human epidermoid carcinoma A431 tumor volume. Human epidermoid carcinoma A431 cells were implanted subcutaneously in female nude mice. PT-NPX with equivalent siRNA of 2 mg/kg was injected intravenously when tumor volume reached 80-100 mm³. Injection schedules are indicated by the arrows below the transverse axis. Treatment with PT-NPX containing human VEGF-siRNA or mouse VEGFR2-siRNA (sense strand: 5′-ggaaggcccauugaguccaacuaca-3′(SEQ ID NO: 327) and antisense strand: 5′-uguaguuggacucaaugggccuucc-3′ (SEQ ID NO: 328)) significantly inhibited tumor growth in comparison with untreated or GFP-siRNA NPX treated controls. The antitumor efficacy was comparable to that of Avastin at 5 mg/kg via i.p. injection. No obvious body weight loss or clinical abnormality in any of the hVEGF-siRNA or mVEGFR2 PT-NPX treated animals was observed.

FIG. 14 is a bar graph showing in vivo knockdown of mouse VEGFR2 mRNA in A549 tumors through systemic treatment with PT-mVEGFR2-siRNA NPX. The in vivo target gene knockdown by PT-siRNA NPX was examined in the A549 xenograft model. Upon establishment of the xenograft tumors, the mice (n=6) were treated i.v. with PT-NPX carrying mVEGFR2-siRNA (sense strand: 5′-ggaaggcccauugaguccaacuaca-3′(SEQ ID NO: 327) and antisense strand: 5′-uguaguuggacucaaugggccuucc-3′ (SEQ ID NO: 328)) or Control-siRNA at 2 mg/kg daily for 3 days. At 24 hours after the last injection, the tumors were removed, and total RNA from tumor tissues were isolated and subjected to a relative quantitative real-time PCR assay. Treatment with PT-mVEGFR2-siRNA NPX resulted in a significant knockdown (between 30-90% in repeated experiments) of mVEGFR2 mRNA in A549 xenografts.

### DETAILED DESCRIPTION OF THE INVENTION

The present invention relates to nucleic acid molecules for modulating the expression of EGFR. In certain embodiments the nucleic acid is ribonucleic acid (RNA). In certain embodiments, the RNA molecules are single or double stranded. In this regard, the nucleic acid based molecules of the present invention, such as siRNA, inhibit or down-regulate expression of EGFR.

The present invention relates to compounds, compositions, and methods for the study, diagnosis, and treatment of traits, diseases and conditions that respond to the modulation of EGFR gene expression and/or activity. The present invention is also directed to compounds, compositions, and methods relating to traits, diseases and conditions that respond to the modulation of expression and/or activity of genes involved in EGFR gene expression pathways or other cellular processes that mediate the maintenance or development of such traits, diseases and conditions. Specifically, the invention relates to double stranded nucleic acid molecules including small nucleic acid molecules, such as short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against EGFR gene expression, including cocktails of such small nucleic acid molecules and nanoparticle formulations of such small nucleic acid molecules. The present invention also relates to small nucleic acid molecules, such as siNA, siRNA, and others that can inhibit the function of endogenous RNA molecules, such as endogenous micro-RNA (miRNA) (e.g, miRNA inhibitors) or endogenous short interfering RNA (siRNA), (e.g., siRNA inhibitors) or that can inhibit the function of RISC (e.g., RISC inhibitors), to modulate EGFR gene expression by interfering with the regulatory function of such endogenous RNAs or proteins associated with such endogenous RNAs (e.g., RISC), including cocktails of such small nucleic acid molecules and nanoparticle formulations of such small nucleic acid molecules. Such small nucleic acid molecules are useful, for example, in providing compositions to prevent, inhibit, or reduce breast, lung, prostate, colorectal, brain, esophageal, bladder, pancreatic, cervical, head and neck, and ovarian cancer, melanoma, lymphoma, glioma, multidrug resistant cancers, and any other cancerous disease and/or other disease states, conditions, or traits associated with EGFR gene expression or activity in a subject or organism.

By “inhibit” or “down-regulate” it is meant that the expression of the EGFR gene, or level of mRNA encoding an EGFR protein, levels of EGFR protein, or activity of EGFR is reduced below that observed in the absence of the nucleic acid molecules of the invention. In one embodiment, inhibition or down-regulation with the nucleic acid molecules of the invention is below that level observed in the presence of an inactive control or attenuated molecule that is able to bind to the same target RNA, but is unable to cleave or otherwise silence that RNA. In another embodiment, inhibition or down-regulation with the nucleic acid molecules of the invention is preferably below that level observed in the presence of, for example, a nucleic acid with scrambled sequence or with mismatches. In another embodiment, inhibition or down-regulation of EGFR with the nucleic acid molecule of the instant invention is greater in the presence of the nucleic acid molecule than in its absence.

By “modulate” is meant that the expression of the EGFR gene, or level of mRNA encoding an EGFR protein, levels of EGFR protein, or activity of EGFR is up-regulated or down-regulated, such that the expression, level, or activity is greater than or less than that observed in the absence of the nucleic acid molecules of the invention.

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short interfering RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein also refers to a double stranded RNA molecule capable of RNA interference “RNAi”, including short interfering RNA “siRNA” (see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914). The dsRNA may be a 25-mer. The dsRNA may be blunt-ended or comprise single-stranded overhangs.

By “gene” it is meant a nucleic acid that encodes an mRNA, for example, nucleic acid sequences include but are not limited to structural genes encoding a polypeptide.

By “a nucleic acid that targets” is meant a nucleic acid as described herein that matches, is complementary to or otherwise binds or specifically hybridizes to and thereby modulates the expression of the gene that comprises the target sequence, or level of mRNA encoding an EGFR protein, levels of EGFR protein, or activity of EGFR.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “RNA interference” or “RNAi” is meant a biological process of inhibiting or down regulating gene expression in a cell as is generally known in the art and which is mediated by short interfering nucleic acid molecules (see for example Zamore and Haley, 2005, Science, 309, 1519-1524; Vaughn and Martienssen, 2005, Science, 309, 1525-1526; Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). In addition, as used herein, the term RNAi is meant to be equivalent to other terms used to describe sequence specific RNA interference, such as post transcriptional gene silencing, translational inhibition, transcriptional inhibition, or epigenetics. For example, siRNA molecules of the invention can be used to epigenetically silence genes at both the post-transcriptional level or prior to transcriptional initiation. In a non-limiting example, epigenetic modulation of gene expression by siRNA molecules of the invention can result from siRNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siRNA molecules of the invention can result from siRNA mediated cleavage of RNA (either coding or non-coding RNA) via RISC, or alternately, translational inhibition as is known in the art. In another embodiment, modulation of gene expression by siRNA molecules of the invention can result from transcriptional inhibition (see for example Janowski et al., 2005, Nature Chemical Biology, 1, 216-222).

In certain embodiments, the nucleic acid inhibitors comprise sequences which are complementary to any known EGFR sequence, including variants thereof that have altered expression and/or activity, particularly variants associated with disease. Variants of EGFR include sequences having 70%, 75%, 80%, 85%, 90%, 91%, 92%, 93%, 94%, 95%, 96%, 97%, 98%, 99% or higher sequence identity to the wild type EGFR sequences, wherein such EGFR variants may demonstrate altered (increased or decreased) tyrosine kinase activity. As would be understood by the skilled artisan, EGFR sequences are available in any of a variety of public sequence databases including GENBANK or SWISSPROT. In one embodiment, the nucleic acid inhibitors (e.g., siRNA) of the invention comprise sequences complimentary to the specific EGFR target sequences provided in SEQ ID NOs: 1-10 and 21-121 (see Tables 1 and 3). Examples of such siRNA molecules also are shown in the Examples and provided in SEQ ID NOs: 11-20 and 122-323 (see Tables 2 and 4).

By “vectors” is meant any nucleic acid- and/or viral-based technique used to deliver a desired nucleic acid.

By “subject” is meant an organism which is a recipient of the nucleic acid molecules of the invention. “Subject” also refers to an organism to which the nucleic acid molecules of the invention can be administered. In certain embodiments, a subject is a mammal or mammalian cells. In further embodiments, a subject is a human or human cell.

Nucleic acids can be synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al, 1998, Biotechnol Bioeng., 61, 33-45, and Brennan, U.S. Pat. No. 6,001,311. The synthesis of nucleic acids makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μM scale protocol with a 2.5 min coupling step for 2′-O-methylated nucleotides and a 45 second coupling step for 2′-deoxy nucleotides. Alternatively, syntheses at the 0.2 μM scale can be performed on a 96-well plate synthesizer, such as the instrument produced by Protogene (Palo Alto, Calif.) with minimal modification to the cycle. A 33-fold excess (60 μL of 0.11 M=6.6 μM) of 2′-O-methyl phosphoramidite and a 105-fold excess of S-ethyl tetrazole (60 μL of 0.25 M=15 μM) can be used in each coupling cycle of 2′-O-methyl residues relative to polymer-bound 5′-hydroxyl. A 22-fold excess (40 μL of 0.11 M=4.4 μM) of deoxy phosphoramidite and a 70-fold excess of S-ethyl tetrazole (40 μL of 0.25 M=10 μM) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5 99%. Other oligonucleotide synthesis reagents for the 394 Applied Biosystems, Inc. synthesizer include; detritylation solution is 3% TCA in methylene chloride (ABI); capping is performed with 16% N-methylimidazole in THF (ABI) and 10% acetic anhydride/10% 2,6-lutidine in THF (ABI); and oxidation solution is 16.9 mM I₂, 49 mM pyridine, 9% water in THF. Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra). There are several examples of modified nucleic acid bases known in the art as summarized by Limbach et al., 1994, Nucleic Acids Res. 22, 2183. Exemplary chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al, International PCT Publication No. WO 93/15187; Uhlman & Peyman). There are several examples of modified nucleic acid bases known in the art as summarized by Limbach et al., 1994, Nucleic Acids Res. 22, 2183. Exemplary chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In certain embodiments, the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45). Those skilled in the art will realize that any nucleic acid can be expressed in eukaryotic cells from the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., 1992, Nucleic Acids Symp. Ser., 27, 15-16; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856).

In another aspect of the invention, nucleic acid molecules of the present invention, such as RNA molecules, are expressed from transcription units (see for example Couture et al., 1996, TIG., 12, 510) inserted into DNA or RNA vectors. The recombinant vectors are preferably DNA plasmids or viral vectors. RNA expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient or subject followed by reintroduction into the patient or subject, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operably linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules, such as ribozymes expressed from such promoters can function in mammalian cells (e.g., Kashani-Sabet et al., 1992, Antisense Res. Dev., 2, 3-15; Ojwang et al., 1992, Proc. Natl. Acad. Sci. USA, 89, 10802-6; Chen et al, 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In yet another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### Methods of Use and Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar; Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. For example, the nucleic acids and compositions of the invention may be administered directly into a tumor. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell. For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes exposes the desired negatively charged nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly(DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The invention also features the use of the composition comprising surface-modified liposomes containing poly(ethylene glycol) lipids (PEG-modified, branched and unbranched or combinations thereof, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present invention includes nucleic acid compositions, such as siRNA compositions, prepared as described in US 2003/0166601. In this regard, in one embodiment, the present invention provides a composition of the siRNA described herein comprising: 1) a core complex comprising the nucleic acid (e.g., siRNA) and polyethyleneimine; and 2) an outer shell moiety comprising NHS-PEG-VS and a targeting moiety.

In certain embodiments of the present invention a targeting moiety as described above is utilized to target the desired siRNA(s) to a cell of interest.

Thus, in certain embodiments, compositions comprising the siRNA molecules of the present invention include at least one targeting moiety, such as a ligand for a cell surface receptor or other cell surface marker that permits highly specific interaction of the composition comprising the siRNA molecule (the “vector”) with the target tissue or cell. More specifically, in one embodiment, the vector preferably will include an unshielded ligand or a shielded ligand. The vector may include two or more targeting moieties, depending on the cell type that is to be targeted. Use of multiple (two or more) targeting moieties can provide additional selectivity in cell targeting, and also can contribute to higher affinity and/or avidity of binding of the vector to the target cell. When more than one targeting moiety is present on the vector, the relative molar ratio of the targeting moieties may be varied to provide optimal targeting efficiency. Methods for optimizing cell binding and selectivity in this fashion are known in the art. The skilled artisan also will recognize that assays for measuring cell selectivity and affinity and efficiency of binding are known in the art and can be used to optimize the nature and quantity of the targeting ligand(s).

Suitable ligands include, but are not limited to: RGD and monoclonal antibodies against receptors on the surface of tumor cells or endothelial cells.

Another example of a targeting moeity is sialyl-Lewis^{x}, where the composition is intended for treating a region of inflammation. Other peptide ligands may be identified using methods such as phage display (F. Bartoli et al., Isolation of peptide ligands for tissue-specific cell surface receptors, in Vector Targeting Strategies for Therapeutic Gene Delivery (Abstracts form Cold Spring Harbor Laboratory 1999 meeting), 1999, p 4) and microbial display (Georgiou et al., Ultra-High Affinity Antibodies from Libraries Displayed on the Surface of Microorganisms and Screened by FACS, in Vector Targeting Strategies for Therapeutic Gene Delivery (Abstracts form Cold Spring Harbor Laboratory 1999 meeting), 1999, p 3.). Ligands identified in this manner are suitable for use in the present invention.

Methods have been developed to create novel peptide sequences that elicit strong and selective binding for target tissues and cells such as “DNA Shuffling” (W. P. C. Stremmer, Directed Evolution of Enzymes and Pathways by DNA Shuffling, in Vector Targeting Strategies for Therapeutic Gene Delivery (Abstracts form Cold Spring Harbor Laboratory 1999 meeting), 1999, p. 5.) and these novel sequence peptides are suitable ligands for the invention. Other chemical forms for ligands are suitable for the invention such as natural carbohydrates which exist in numerous forms and are a commonly used ligand by cells (Kraling et al., Am. J. Path., 1997, 150, 1307) as well as novel chemical species, some of which may be analogues of natural ligands such as D-amino acids and peptidomimetics and others which are identified through medicinal chemistry techniques such as combinatorial chemistry (P. D. Kassner et al., Ligand Identification via Expression (LIVEθ): Direct selection of Targeting Ligands from Combinatorial Libraries, in Vector Targeting Strategies for Therapeutic Gene Delivery (Abstracts form Cold Spring Harbor Laboratory 1999 meeting), 1999, p 8.).

In a further embodiment, the present invention includes nucleic acid compositions prepared for delivery as described in U.S. Pat. No. 7,163,695, U.S. Pat. No. 7,070,807 and U.S. Pat. No. 6,692,911. In this regard, in one embodiment, the present invention provides a nucleic acid of the present invention in a composition comprising the histidine-lysine copolymers (also referred to herein as PolyTran™) as described in U.S. Pat. Nos. 7,163,695, 7,070,807 and 6,692,911 either alone or in combination with PEG (e.g., branched or unbranched PEG or a mixture of both) or in combination with PEG and a targeting moiety.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington: The Science and Practice of Pharmacy, 20th Edition. Baltimore, Md.: Lippincott Williams & Wilkins, 2000. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

The nucleic acid compositions of the invention can be used in combination with other nucleic acid compositions that target the same or different areas of the target gene (e.g., EGFR), or that target other genes of interest. The nucleic acid compositions of the invention can also be used in combination with any of a variety of treatment modalities, such as chemotherapy, radiation therapy, or small molecule regimens.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.01 mg to about 140 mg per kilogram of body weight per day are useful in the treatment of the disease conditions described herein (about 0.5 mg to about 7 g per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers.

The nucleic acid molecules of the instant invention may be used in compositions comprising multiple nucleic acid molecules (siRNAs) targeting different target sequences within the EGFR gene or targeting sequences within other genes.

The nucleic acid molecules of the instant invention, individually, or in combination or in conjunction with other drugs, can be used to treat diseases or conditions associated with altered expression and/or activity of EGFR. Thus, the small nucleic acid molecules described herein are useful, for example, in providing compositions to prevent, inhibit, or reduce breast, lung, prostate, colorectal, brain, esophageal, bladder, pancreatic, cervical, head and neck, and ovarian cancer, melanoma, lymphoma, glioma, multidrug resistant cancers, and any other cancerous diseases and/or other disease states, conditions, or traits associated with EGFR gene expression or activity in a subject or organism.

The nucleic acid molecules of the instant invention, individually, or in combination or in conjunction with other drugs, can also be used to prevent diseases or conditions associated with altered activity and/or expression of EGFR in individuals that are suspected of being at risk for developing such a disease or condition. For example, to treat or prevent a disease or condition associated with the expression levels of EGFR, the subject having the disease or condition, or suspected of being at risk for developing the disease or condition, can be treated, or other appropriate cells can be treated, as is evident to those skilled in the art, individually or in combination with one or more drugs under conditions suitable for the treatment. Thus, the present invention provides methods for treating or preventing diseases or conditions which respond to the modulation of EGFR expression comprising administering to a subject in need thereof an effective amount of a composition comprising one or more of the nucleic acid molecules of the invention, such as those set forth in SEQ ID NOs: 11-20 and 122-323. In one embodiment, the present invention provides methods for treating or preventing diseases associated with expression of EGFR comprising administering to a subject in need thereof an effective amount of any one or more of the nucleic acid molecules of the invention, such as those provided in SEQ ID NOs: 11-20 and 122-323, such that the expression of EGFR in the subject is down-regulated, thereby treating or preventing the disease associated with expression of EGFR. In this regard, the compositions of the invention can be used in methods for treating or preventing breast, lung, prostate, colorectal, brain, esophageal, bladder, pancreatic, cervical, head and neck, meningioma, kidney, endometrial, and ovarian cancer, melanoma, lymphoma, glioblastoma, multidrug resistant cancers, and any other cancerous diseases, or other conditions which respond to the modulation of EGFR expression.

In a further embodiment, the nucleic acid molecules of the invention, such as siRNA, antisense or ribozymes, can be used in combination with other known treatments to treat conditions or diseases discussed herein. For example, the described molecules can be used in combination with one or more known therapeutic or diagnostic agents to treat breast, lung, prostate, colorectal, brain, esophageal, bladder, pancreatic, cervical, head and neck, meningioma, kidney, endometrial, and ovarian cancer, melanoma, lymphoma, glioblastoma, multidrug resistant cancers, and any other cancerous diseases or other conditions which respond to the modulation of EGFR expression. In another embodiment, the nucleic acid molecules of the present invention can be used to treat lung cancer, kidney cancer, pancreas cancer, breast cancer, head and neck cancer, stomach cancer or colon cancer.

In certain embodiments, therapeutic agents that may be used in conjunction with the siRNA molecules of the present invention to treat a cancer as described herein may include agents such as, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the RNA molecules of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan.

In a further embodiment, the RNA molecules of the present invention can be modified according to US patent publication 2005/0186586, 2005/0181382 and/or 2006/0134787 by introducing one or more mismatch(s) into siRNA duplex by modifying the sequence of sense strand of siRNA, to, among other things, decrease the stability of the 5′ antisense end of the molecule to preferentially guide the proper strand into the RISC complex or reduce off target effect. Additionally, the RNA molecules of the present invention can be modified according to US2005/0037988 by introducing wobble base pair (G/U) between antisense strand of siRNA and its complementary target mRNA, to, among other things, increase RISC turnover.

Compositions and methods are known in the art for identifying subjects having, or suspected of being at risk for having the diseases or disorders associated with expression of EGFR as described herein.

### EXAMPLES

### Example 1

### Antitumor Efficacy from Systemically Delivered hEGFR-siRNA Formulated with PolyTran™ in A431 Model

FIG. 3. Tumor inhibition effect of hEGFR-siRNA-PolyTran™ NPX on A431 tumor xenografts

Antitumor efficacy of PolyTran™ (PT-NPX) (FIG. 6) carrying hEGFR-siRNA was determined in A431 xenograft model. Human epidermoid carcinoma A431 cells (5×10⁶ cells per mouse) were implanted subcutaneously into female nude mice. Mice bearing established tumors were treated with intravenous administration of PolyTran NPX carrying hEGFR-siRNA (2 mg/kg, 1:1 mixture of hEGFR-25-1 and hEGFR-25-2) every other day for 6 times started on Day 4 post tumor cells implantation, when tumor size was around 80-100 mm³. PolyTran-siRNA NPX was prepared by mixing PolyTran peptide with siRNA at 3:1 ratio (w/w) and the particle size of NPX is around 100 nm. Treatment controls included no treatment (untreated) and Erlotinib (Tarceva™, a FDA approved EGFR inhibitor) which was daily administered orally at 100 mg/kg for 6 days. Tumor size was measured every other day before administration of PT-siRNA NPX.

Treatment with PT-NPX carrying human EGFR siRNA at 2 mg/kg significantly inhibited A431 tumor growth in comparison with untreated control; and the inhibition effect was more profound than the Tarceva™ treatment control.

### Example 2

### Antitumor Efficacy from Systemically Delivered PT-siRNA NPX is hEGFR-siRNA Specific and Requires Formulation of PT-NPX

FIG. 4. Inhibition of A431 tumor growth by PT-EGFR-siRNA NPX is hEGFR-siRNA specific and requires formulation of PT-siRNA NPX

To confirm that the anti-tumor efficacy in Sample 1 is hEGFR-siRNA specific and requires formulation of siRNA with PT-NPX, PolyTran™ (PT-NPX) carrying hEGFR-siRNA or negative control-siRNA, as well as the PolyTran peptide alone or hEGFR-siRNA alone, were tested in A431 xenograft model. Human epidermoid carcinoma A431 cells (5×10⁶ cells per mouse) were implanted subcutaneously into female nude mice. Mice bearing established tumors were treated with intravenous administration of PolyTran NPX carrying hEGFR-siRNA (2 mg/kg, 1:1 mixture of hEGFR-25-1 and hEGFR-25-2) or negative control-siRNA (2 mg/kg), or hEGFR-siRNA alone (2 mg/kg, 1:1 mixture of hEGFR-25-1 and hEGFR-25-2), or PolyTran peptide alone (6 mg/kg peptide) every other day for 4 times started on Day 5 post tumor cells implantation, when tumor size was around 80-100 mm³. PolyTran-siRNA NPX was prepared by mixing PolyTran peptide with siRNA at 3:1 ratio (w/w) and the particle size of NPX is around 100 nm. Treatment controls included no treatment (untreated). Tumor size was measured every other day before administration of testing articles.

Only the treatment with PT-NPX carrying human EGFR siRNA at 2 mg/kg significantly inhibited A431 tumor growth in comparison with untreated control. All other treatment groups include PT-NPX carrying control-siRNA, hEGFR-siRNA alone, or PolyTran peptide, did not inhibit A431 tumor growth.

### Example 3

### Antitumor Efficacy from Systemically Delivered hEGFR-siRNA Formulated with PolyTran™ in A549 Model

FIG. 5. Tumor inhibition effect of hEGFR-siRNA-PolyTran™ NPX on A549 tumor xenografts

In addition to A431 model, the antitumor efficacy of PolyTran™ (PT-NPX) carrying hEGFR-siRNA was determined in A549 xenograft model. Human Non-small Cell Lung Cancer (NSCLC) A549 cells (5×10⁶ cells per mouse) were implanted subcutaneously into female nude mice. Mice bearing established tumors were treated with intravenous administration of PolyTran NPX carrying hEGFR-siRNA (2 mg/kg, 1:1 mixture of hEGFR-25-1 and hEGFR-25-2) or negative control-siRNA (2 mg/kg) every other day for 6 times started on Day 9 post tumor cells implantation, when tumor size was around 80-100 mm³. PolyTran-siRNA NPX was prepared by mixing PolyTran peptide with siRNA at 3:1 ratio (w/w) and the particle size of NPX is around 100 nm. Treatment controls included no treatment (untreated) and Erlotinib (Tarceva™, a FDA approved EGFR inhibitor) which was daily administered orally at 100 mg/kg for 6 days. Tumor size was measured every other day before administration of PT-siRNA NPX.

Treatment with PT-NPX carrying human EGFR siRNA at 2 mg/kg significantly inhibited A549 tumor growth in comparison with untreated control; and the inhibition effect was more profound than the Tarceva™ treatment control. The PT-NPX carrying control-siRNA did not have inhibition effect on A549 tumor growth.

### Example 4

### siRNA Molecules Inhibit Human EGFR Expression

Human EGFR 25-mer siRNA molecules were designed using the publicly available sequence for the human EGFR gene (NM_{—}005228). Table 1 shows the target sequence of hEGFR-siRNA candidates.

TABLE 1
Target DNA Sequence of hEGFR-siRNA Candidates

| SEQ ID | hEGFR start | | | |
|---|---|---|---|---|
| NO: | position | DNA Sequence | Region | GC % |
| 1 | 528 | CACAGTGGAGCGAATTCCTTTGGAA | ORF | 48.0 |
| 2 | 1246 | CGCAAAGTGTGTAACGGAATAGGTA | ORF | 44.0 |
| 3 | 2438 | GGATCCCAGAAGGTGAGAAAGTTAA | ORF | 44.0 |
| 4 | 2789 | CGCAGCATGTCAAGATCACAGATTT | ORF | 44.0 |
| 5 | 2858 | CAGAAGGAGGCAAAGTGCCTATCAA | ORF | 48.0 |
| 6 | 2874 | GCCTATCAAGTGGATGGCATTGGAA | ORF | 48.0 |
| 7 | 3214 | CCAAGTCCTACAGACTCCAACTTCT | ORF | 48.0 |
| 8 | 3355 | TCTCTGAGTGCAACCAGCAACAATT | ORF | 44.0 |
| 9 | 3435 | CAGCTTCTTGCAGCGATACAGCTCA | ORF | 52.0 |
| 10 | 3784 | GAAGCCAAGCCAAATGGCATCTTTA | ORF | 44.0 |

Candidate siRNA molecules were synthesized using standard techniques. siRNA candidates are shown in Table 2.

TABLE 2
hEGFR siRNA Molecules

| | | | SEQ |
|---|---|---|---|
| | | siRNA sequence | ID |
| ID NO: | Name | (sense strand/antisense strand) | NO: |
| 07-25- | hEGFR-25-1 | 5′-r(CACAGUGGAGCGAAUUCCUUUGGAA)-3′ | 11 |
| 001 | | 3′--(GUGUCACCUCGCUUAAGGAAACCUU)r-5′ | 12 |
| 07-25- | hEGFR-25-2 | 5′-r(CGCAAAGUGUGUAACGGAAUAGGUA)-3′ | 13 |
| 002 | | 3′--(GCGUUUCACACAUUGCCUUAUCCAU)r-5′ | 14 |
| 07-25- | hEGFR-25-3 | 5′-r(GGAUCCCAGAAGGUGAGAAAGUUAA)-3′ | 15 |
| 003 | | 3′--(CCUAGGGUCUUCCACUCUUUCAAUU)r-5′ | 16 |
| 07-25- | hEGFR-25-4 | 5′ -r(CGCAGCAUGUCAAGAUCACAGAUUU)-3′ | 17 |
| 004 | | 3′--(GCGUCGUACAGUUCUAGUGUCUAAA)r-5′ | 18 |
| 07-25- | hEGFR-25-5 | 5′-r(CCAAGUCCUACAGACUCCAACUUCU)-3′ | 19 |
| 00 | | 3′--(GGUUCAGGAUGUCUGAGGUUGAAGA)r-5′ | 20 |

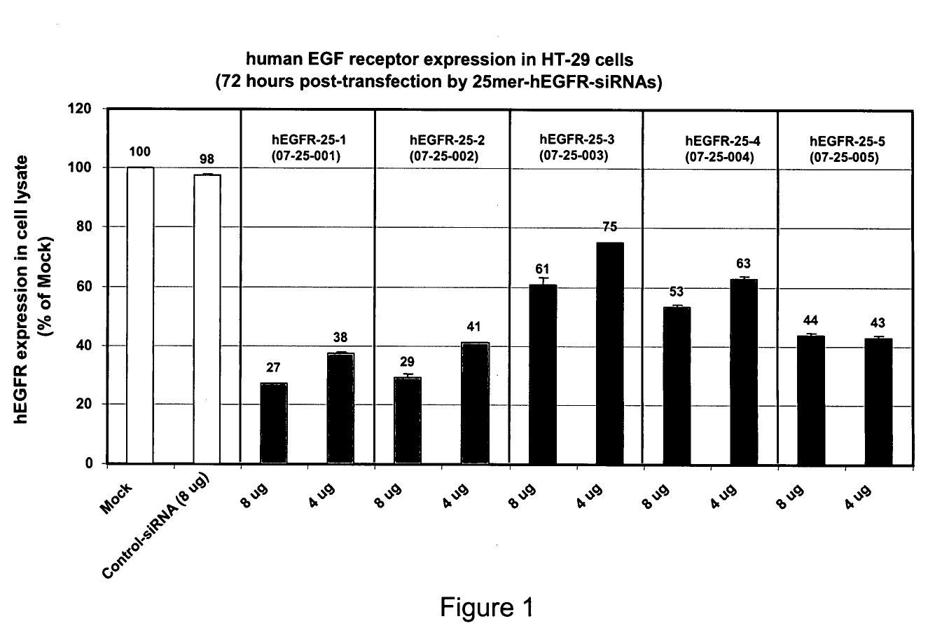

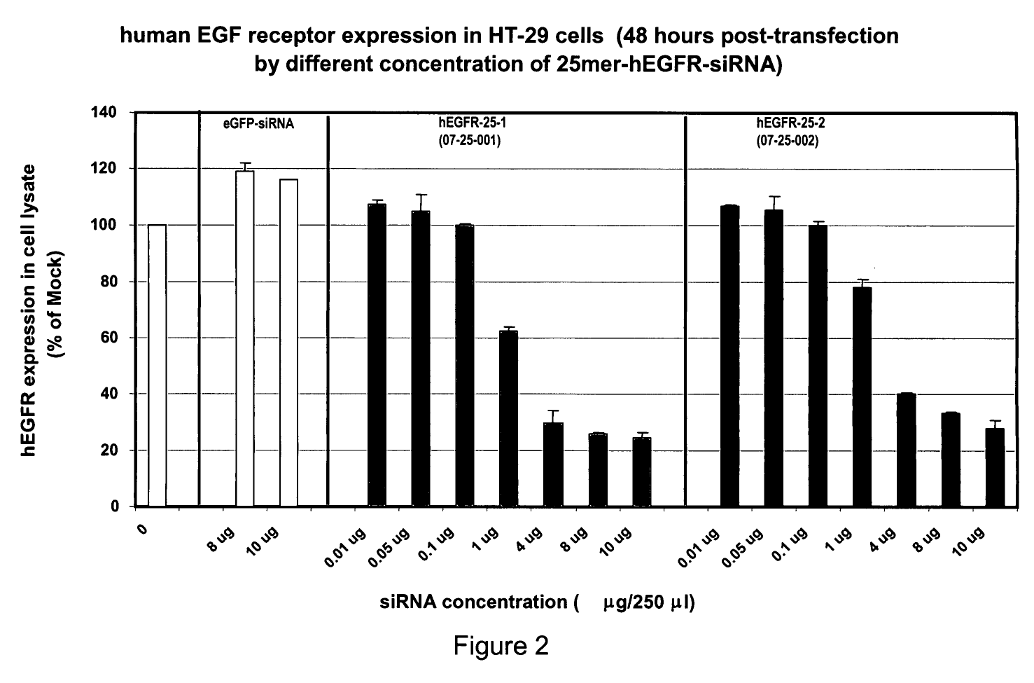

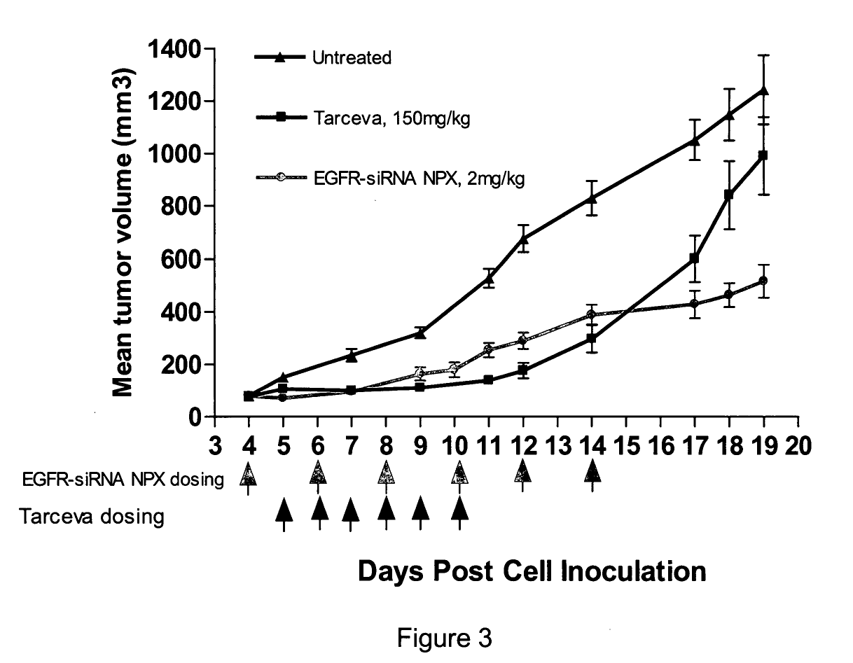

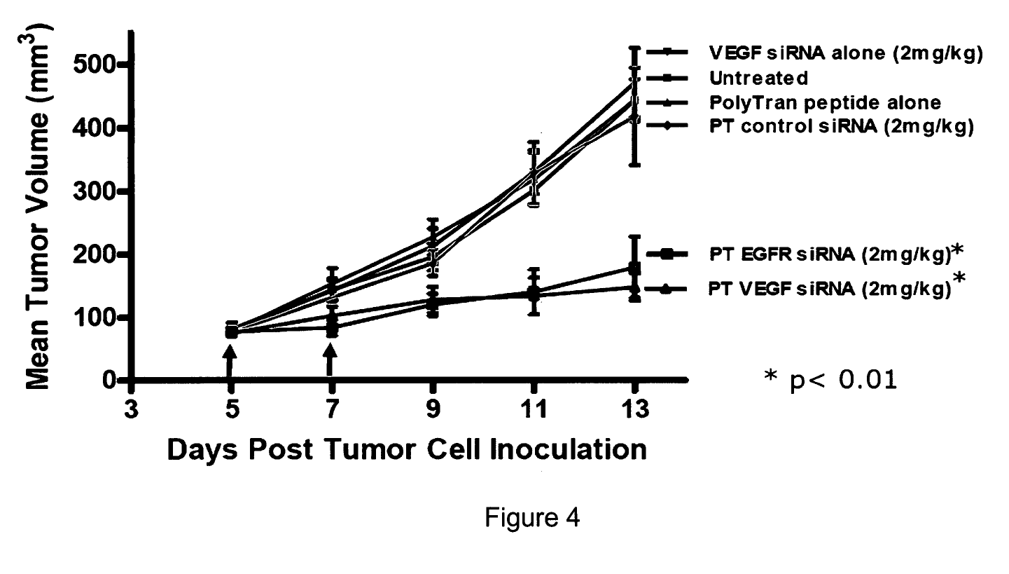

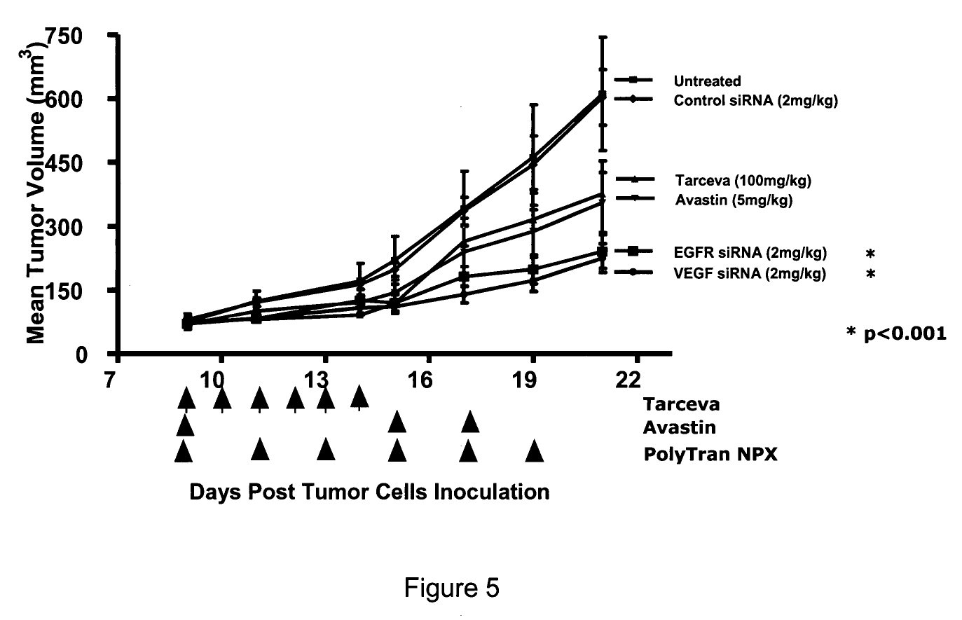

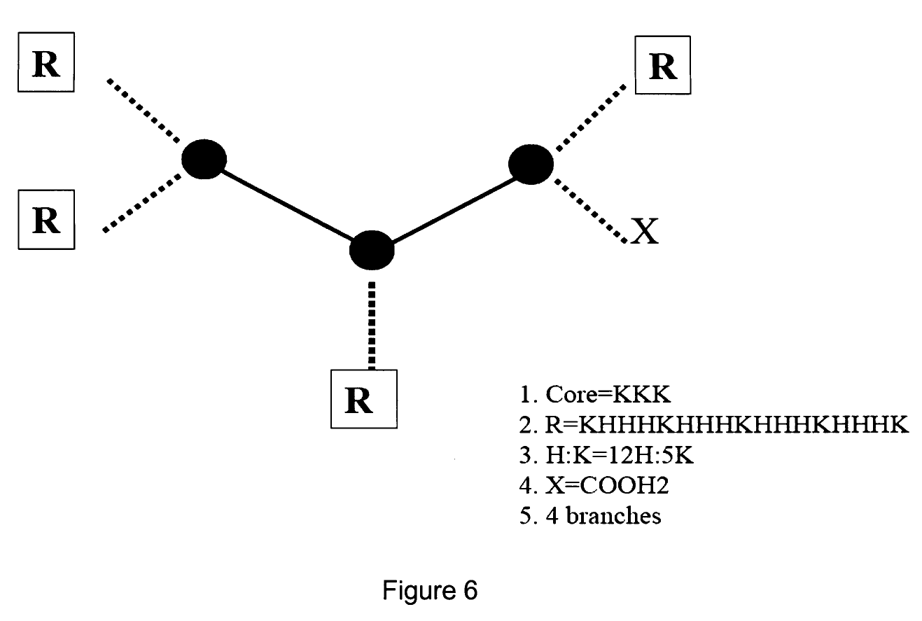

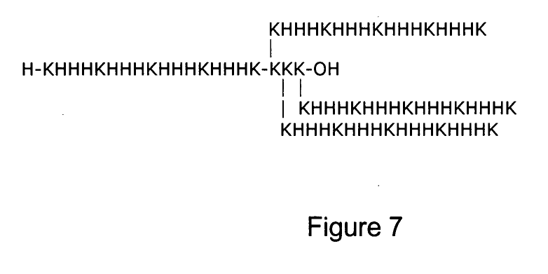

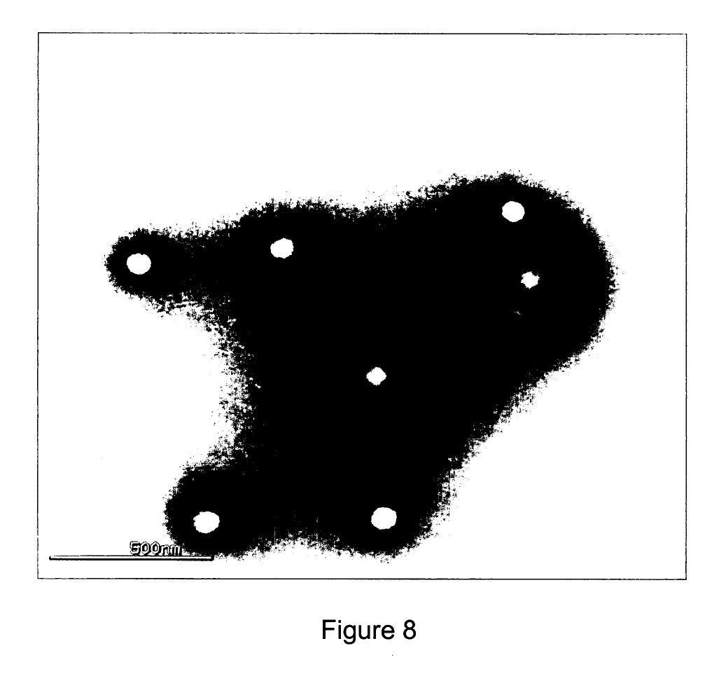

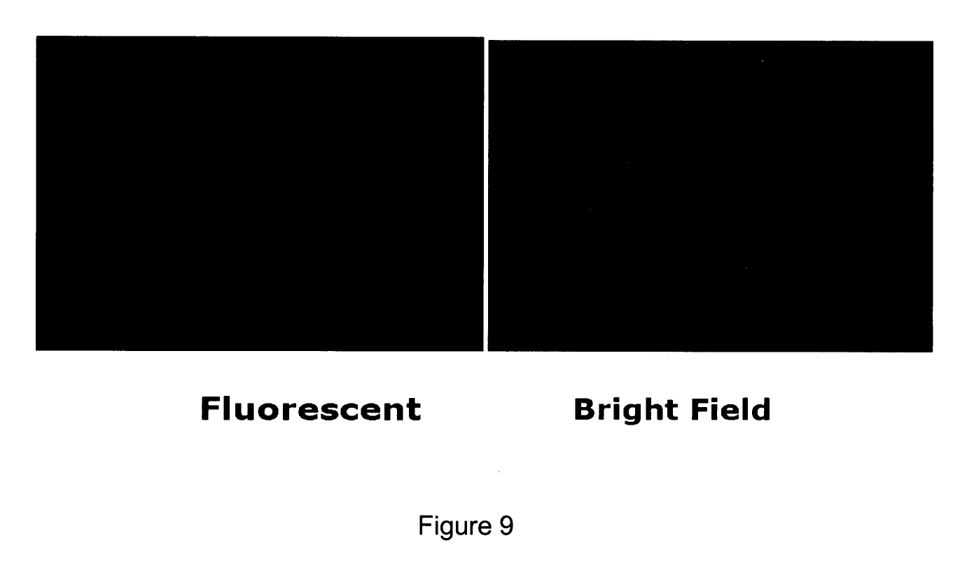

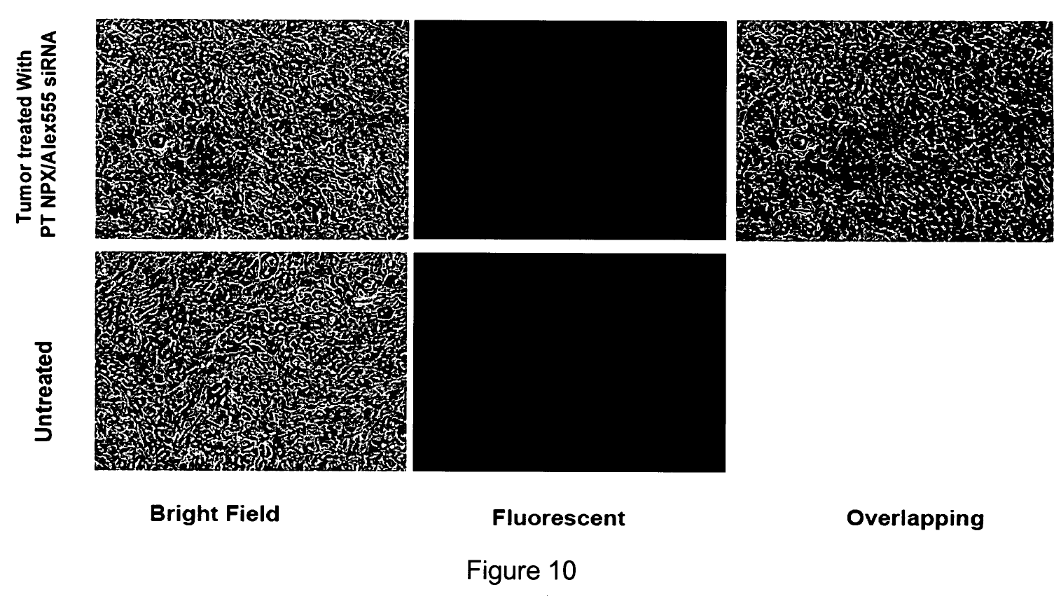

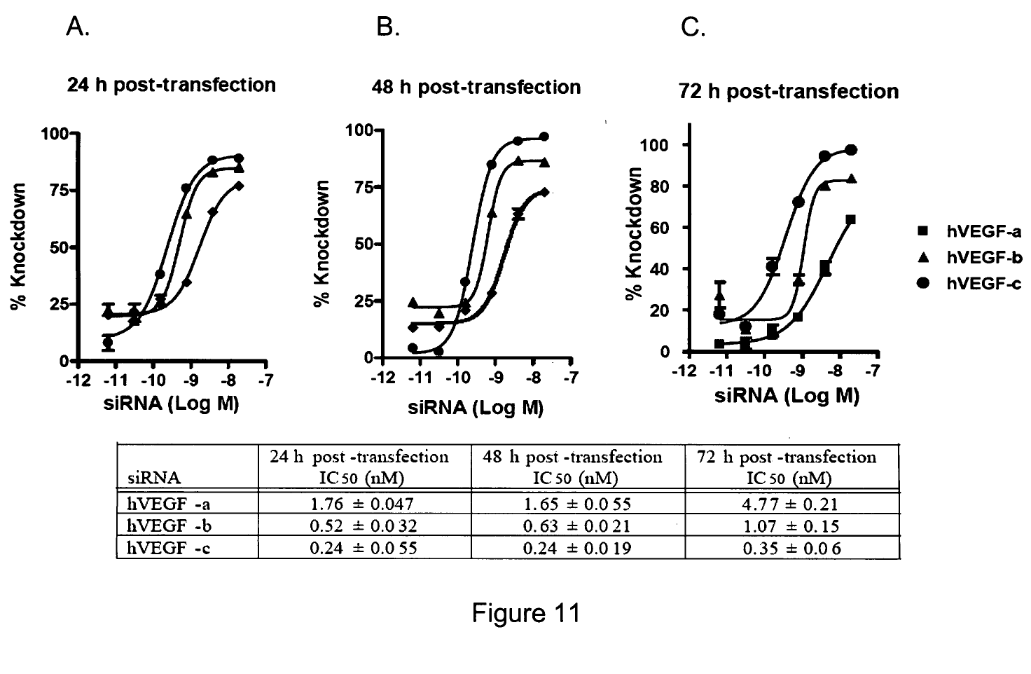

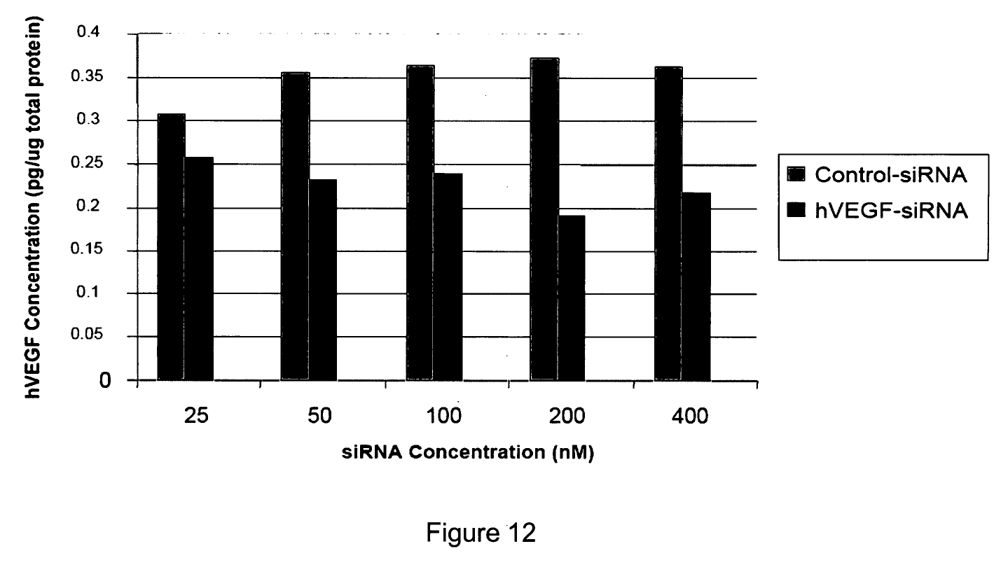

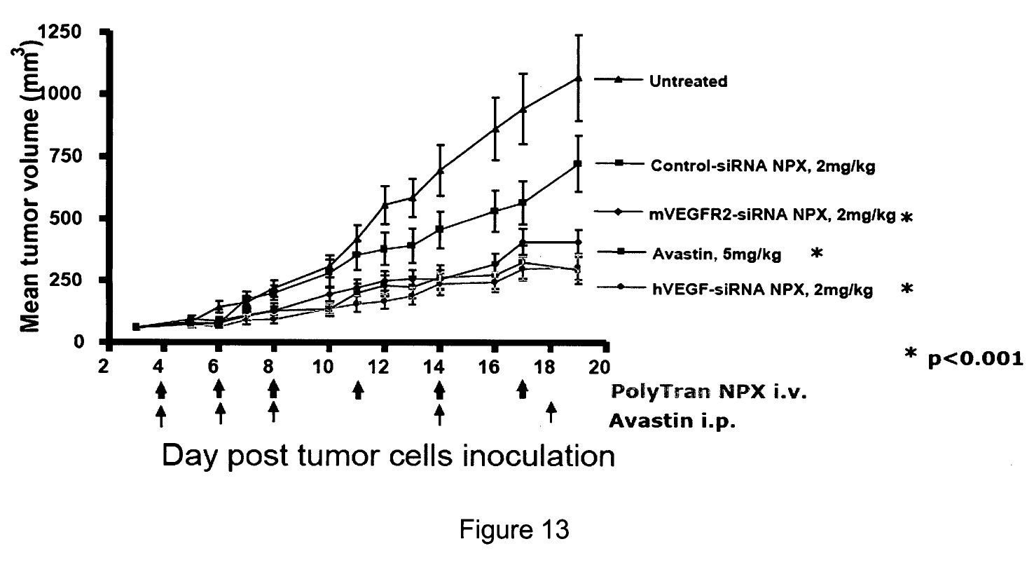

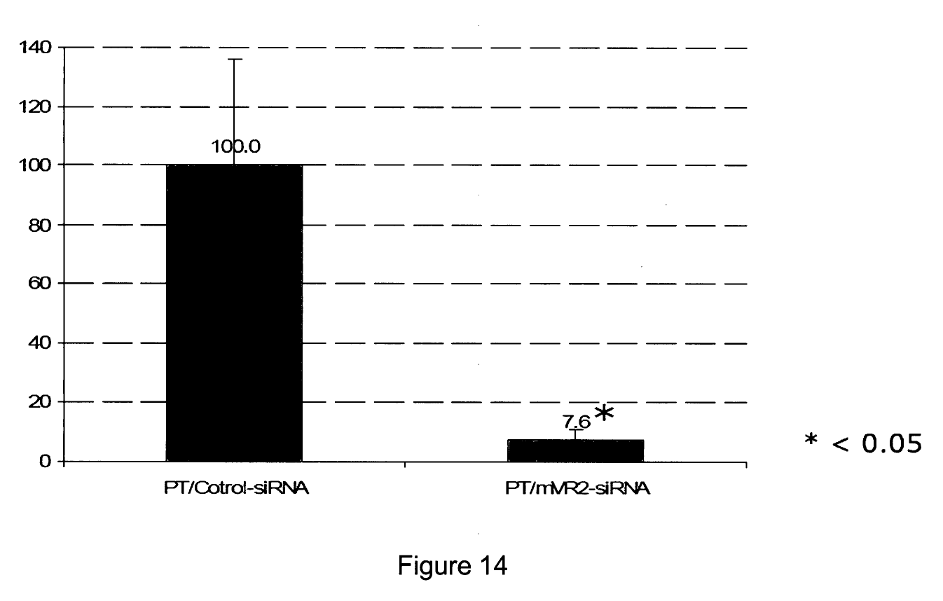

The above candidates were screened in vitro for knockdown activity of the hEGFR gene. HT-29 cells were transfected using electroporation with the siRNA candidates (Table 2) and hEGFR protein expression was assayed at 72 hours post-transfection using a commercially available ELISA kit (see FIG. 1).

Two siRNA candidates, hEGFR-25-1 and hEGFR-25-2, were further tested for activity in a dose titration experiment. As shown in FIG. 2, these two hEGFR siRNA candidates inhibited hEGFR expression in a dose-dependent manner.

In summary, this experiment shows successful inhibition of EGFR expression by numerous siRNA candidates. These siRNA candidates can be used for the treatment of diseases.

### Example 5

### siRNA Candidate Molecules for the Inhibition of Human EGFR Expression

Human EGFR 25-mer siRNA molecules were designed using a tested algorithm and using the publicly available sequences for human EGFR gene (NM_{—}005228). Table 3 shows the target sequence of hEGFR-siRNA candidates.

TABLE 3
Target DNA Sequence of hEGFR-siRNA Candidates

| SEQ | hEGFR | | | |
|---|---|---|---|---|
| ID | start | | | GC |
| NO: | postion | DNA Sequence | Region | % |
| 21 | 91 | 5′TGCCAAGGCACGAGTAACAAGCTCA3′ | 5′UTR | 52 |
| 22 | 98 | 5′GCACGAGTAACAAGCTCACGCAGTT3′ | 5′UTR | 52 |
| 23 | 194 | 5′TGGAAATTACCTATGTGCAGAGGAA3′ | 5′UTR | 40 |
| 24 | 195 | 5′GGAAATTACCTATGTGCAGAGGAAT3′ | 5′UTR | 40 |
| 25 | 196 | 5′GAAATTACCTATGTGCAGAGGAATT3′ | 5′UTR | 36 |
| 26 | 201 | 5′TACCTATGTGCAGAGGAATTATGAT3′ | 5′UTR | 36 |
| 27 | 203 | 5′CCTATGTGCAGAGGAATTATGATCT3′ | 5′UTR | 40 |
| 28 | 209 | 5′TGCAGAGGAATTATGATCTTTCCTT3′ | 5′UTR | 36 |
| 29 | 211 | 5′CAGAGGAATTATGATCTTTCCTTCT3′ | 5′UTR | 36 |
| 30 | 346 | 5′TCCTATGCCTTAGCAGTCTTATCTA3′ | ORF | 40 |
| 31 | 347 | 5′CCTATGCCTTAGCAGTCTTATCTAA3′ | ORF | 40 |
| 32 | 353 | 5′CCTTAGCAGTCTTATCTAACTATGA3′ | ORF | 36 |
| 33 | 494 | 5′GGGACATAGTCAGCAGTGACTTTCT3′ | ORF | 48 |
| 34 | 500 | 5′TAGTCAGCAGTGACTTTCTCAGCAA3′ | ORF | 44 |
| 35 | 887 | 5′CCCGTAATTATGTGGTGACAGATCA3′ | ORF | 44 |
| 36 | 888 | 5′CCGTAATTATGTGGTGACAGATCAC3′ | ORF | 44 |
| 37 | 966 | 5′CGTCCGCAAGTGTAAGAAGTGCGAA3′ | ORF | 52 |
| 38 | 972 | 5′CAAGTGTAAGAAGTGCGAAGGGCCT3′ | ORF | 52 |
| 39 | 994 | 5′CCTTGCCGCAAAGTGTGTAACGGAA3′ | ORF | 52 |
| 40 | 999 | 5′CCGCAAAGTGTGTAACGGAATAGGT3′ | ORF | 48 |
| 41 | 1000 | 5′CGCAAAGTGTGTAACGGAATAGGTA3′ | ORF | 44 |
| 42 | 1001 | 5′GCAAAGTGTGTAACGGAATAGGTAT3′ | ORF | 40 |
| 43 | 1002 | 5′CAAAGTGTGTAACGGAATAGGTATT3′ | ORF | 36 |
| 44 | 1043 | 5′CACTCTCCATAAATGCTACGAATAT3′ | ORF | 36 |
| 45 | 1156 | 5′CCTCTGGATCCACAGGAACTGGATA3′ | ORF | 52 |
| 46 | 1160 | 5′TGGATCCACAGGAACTGGATATTCT3′ | ORF | 44 |
| 47 | 1250 | 5′TCCATGCCTTTGAGAACCTAGAAAT3′ | ORF | 40 |
| 48 | 1284 | 5′CAGGACCAAGCAACATGGTCAGTTT3′ | ORF | 48 |
| 49 | 1309 | 5′TCTCTTGCAGTCGTCAGCCTGAACA3′ | ORF | 52 |
| 50 | 1337 | 5′CATCCTTGGGATTACGCTCCCTCAA3′ | ORF | 52 |
| 51 | 1355 | 5′CCCTCAAGGAGATAAGTGATGGAGA3′ | ORF | 48 |
| 52 | 1356 | 5′CCTCAAGGAGATAAGTGATGGAGAT3′ | ORF | 44 |
| 53 | 1358 | 5′TCAAGGAGATAAGTGATGGAGATGT3′ | ORF | 40 |
| 54 | 1361 | 5′AGGAGATAAGTGATGGAGATGTGAT3′ | ORF | 40 |
| 55 | 1362 | 5′GGAGATAAGTGATGGAGATGTGATA3′ | ORF | 40 |
| 56 | 1363 | 5′GAGATAAGTGATGGAGATGTGATAA3′ | ORF | 36 |
| 57 | 1636 | 5′CCAAGGGAGTTTGTGGAGAACTCTG3′ | ORF | 52 |
| 58 | 1642 | 5′GAGTTTGTGGAGAACTCTGAGTGCA3′ | ORF | 48 |
| 59 | 1727 | 5′CAGACAACTGTATCCAGTGTGCCCA3′ | ORF | 52 |
| 60 | 1735 | 5′TGTATCCAGTGTGCCCACTACATTG3′ | ORF | 48 |
| 61 | 1861 | 5′CATCCAAACTGCACCTACGGATGCA3′ | ORF | 52 |
| 62 | 2171 | 5′GCACGGTGTATAAGGGACTCTGGAT3′ | ORF | 52 |
| 63 | 2222 | 5′CCGTCGCTATCAAGGAATTAAGAGA3′ | ORF | 44 |
| 64 | 2223 | 5′CGTCGCTATCAAGGAATTAAGAGAA3′ | ORF | 40 |
| 65 | 2226 | 5′CGCTATCAAGGAATTAAGAGAAGCA3′ | ORF | 40 |
| 66 | 2232 | 5′CAAGGAATTAAGAGAAGCAACATCT3′ | ORF | 36 |
| 67 | 2272 | 5′GAAATCCTCGATGAAGCCTACGTGA3′ | ORF | 48 |
| 68 | 2542 | 5′CCGCAGCATGTCAAGATCACAGATT3′ | ORF | 48 |
| 69 | 2543 | 5′CGCAGCATGTCAAGATCACAGATTT3′ | ORF | 44 |
| 70 | 2608 | 5′CATGCAGAAGGAGGCAAAGTGCCTA3′ | ORF | 52 |
| 71 | 2612 | 5′CAGAAGGAGGCAAAGTGCCTATCAA3′ | ORF | 48 |
| 72 | 2624 | 5′AAGTGCCTATCAAGTGGATGGCATT3′ | ORF | 44 |
| 73 | 2628 | 5′GCCTATCAAGTGGATGGCATTGGAA3′ | ORF | 48 |
| 74 | 2629 | 5′CCTATCAAGTGGATGGCATTGGAAT3′ | ORF | 44 |
| 75 | 2634 | 5′CAAGTGGATGGCATTGGAATCAATT3′ | ORF | 40 |
| 76 | 2803 | 5′CAGCCACCCATATGTACCATCGATG3′ | ORF | 52 |
| 77 | 2807 | 5′CACCCATATGTACCATCGATGTCTA3′ | ORF | 44 |
| 78 | 2815 | 5′TGTACCATCGATGTCTACATGATCA3′ | ORF | 40 |
| 79 | 2858 | 5′TAGACGCAGATAGTCGCCCAAAGTT3′ | ORF | 48 |
| 80 | 2968 | 5′CCAAGTCCTACAGACTCCAACTTCT3′ | ORF | 48 |
| 81 | 2969 | 5′CAAGTCCTACAGACTCCAACTTCTA3′ | ORF | 44 |
| 82 | 2981 | 5′ACTCCAACTTCTACCGTGCCCTGAT3′ | ORF | 52 |
| 83 | 3109 | 5′TCTCTGAGTGCAACCAGCAACAATT3′ | ORF | 44 |
| 84 | 3129 | 5′CAATTCCACCGTGGCTTGCATTGAT3′ | ORF | 48 |
| 85 | 3134 | 5′CCACCGTGGCTTGCATTGATAGAAA3′ | ORF | 48 |
| 86 | 3135 | 5′CACCGTGGCTTGCATTGATAGAAAT3′ | ORF | 44 |
| 87 | 3145 | 5′TGCATTGATAGAAATGGGCTGCAAA3′ | ORF | 40 |
| 88 | 3146 | 5′GCATTGATAGAAATGGGCTGCAAAG3′ | ORF | 44 |
| 89 | 3189 | 5′CAGCTTCTTGCAGCGATACAGCTCA3′ | ORF | 52 |
| 90 | 3262 | 5′CCAGTGCCTGAATACATAAACCAGT3′ | ORF | 44 |
| 91 | 3431 | 5′CCACCTGTGTCAACAGCACATTCGA3′ | ORF | 52 |
| 92 | 3472 | 5′GCCCAGAAAGGCAGCCACCAAATTA3′ | ORF | 52 |
| 93 | 3473 | 5′CCCAGAAAGGCAGCCACCAAATTAG3′ | ORF | 52 |
| 94 | 3537 | 5′GGAAGCCAAGCCAAATGGCATCTTT3′ | ORF | 48 |
| 95 | 3538 | 5′GAAGCCAAGCCAAATGGCATCTTTA3′ | ORF | 44 |
| 96 | 3539 | 5′AAGCCAAGCCAAATGGCATCTTTAA3′ | ORF | 40 |
| 97 | 3552 | 5′TGGCATCTTTAAGGGCTCCACAGCT3′ | ORF | 52 |
| 98 | 3555 | 5′CATCTTTAAGGGCTCCACAGCTGAA3′ | ORF | 48 |
| 99 | 3634 | 5′CCACGGAGGATAGTATGAGCCCTAA3′ | ORF | 52 |
| 100 | 3635 | 5′CACGGAGGATAGTATGAGCCCTAAA3′ | ORF | 48 |
| 101 | 3846 | 5′TACAGAAACGCATCCAGCAAGAATA3′ | ORF | 40 |
| 102 | 4327 | 5′TGATGGACCAGTGGTTTCCAGTCAT3′ | 3′UTR | 48 |
| 103 | 4335 | 5′CAGTGGTTTCCAGTCATGAGCGTTA3′ | 3′UTR | 48 |
| 104 | 4432 | 5′CAGCAAGAGAGGATGACACATCAAA3′ | 3′UTR | 44 |
| 105 | 4471 | 5′CCAGCCCACATTGGATTCATCAGCA3′ | 3′UTR | 52 |
| 106 | 4472 | 5′CAGCCCACATTGGATTCATCAGCAT3′ | 3′UTR | 48 |
| 107 | 4474 | 5′GCCCACATTGGATTCATCAGCATTT3′ | 3′UTR | 44 |
| 108 | 4510 | 5′CCACAGCTGAGAATGTGGAATACCT3′ | 3′UTR | 48 |
| 109 | 4511 | 5′CACAGCTGAGAATGTGGAATACCTA3′ | 3′UTR | 44 |
| 110 | 4570 | 5′TCTCCTAATTTGAGGCTCAGATGAA3′ | 3′UTR | 40 |
| 111 | 4581 | 5′GAGGCTCAGATGAAATGCATCAGGT3′ | 3′UTR | 48 |
| 112 | 4879 | 5′CAGGTGCGAATGACAGTAGCATTAT3′ | 3′UTR | 44 |
| 113 | 4884 | 5′GCGAATGACAGTAGCATTATGAGTA3′ | 3′UTR | 40 |
| 114 | 4892 | 5′CAGTAGCATTATGAGTAGTGTGGAA3′ | 3′UTR | 40 |
| 115 | 4897 | 5′GCATTATGAGTAGTGTGGAATTCAG3′ | 3′UTR | 40 |
| 116 | 4905 | 5′AGTAGTGTGGAATTCAGGTAGTAAA3′ | 3′UTR | 36 |
| 117 | 5079 | 5′TGTGCCCTGTAACCTGACTGGTTAA3′ | 3′UTR | 48 |
| 118 | 5337 | 5′CCTGACTGGTTAACAGCAGTCCTTT3′ | 3′UTR | 48 |
| 119 | 5340 | 5′GACTGGTTAACAGCAGTCCTTTGTA3′ | 3′UTR | 44 |
| 120 | 5350 | 5′CAGCAGTCCTTTGTAAACAGTGTTT3′ | 3′UTR | 40 |
| 121 | 5442 | 5′CAGCCTACAGTTATGTTCAGTCACA3′ | 3′UTR | 44 |

hEGFR candidate siRNA molecules are shown in Table 4 below and are set forth in SEQ ID NOs: 122-323.

TABLE 4
hEGFR Candidate siRNA Molecules

| | | | SEQ |
|---|---|---|---|
| | | siRNA sequence | ID |
| ID NO: | Name | (sense strand/antisense strand) | NO: |
| 07-25- | hEGFR- | 5′-r(UGCCAAGGCACGAGUAACAAGCUCA)-3′ | 122 |
| 021 | 25-21 | 3′-(ACGGUUCCGUGCUCAUUGUUCGAGU)r-5′ | 123 |
| 07-25- | hEGFR- | 5′-r(GCACGAGUAACAAGCUCACGCAGUU)-3′ | 124 |
| 022 | 25-22 | 3′-(CGUGCUCAUUGUUCGAGUGCGUCAA)r-5′ | 125 |
| 07-25- | hEGFR- | 5′-r(UGGAAAUUACCUAUGUGCAGAGGAA)-3′ | 126 |
| 023 | 25-23 | 3′-(ACCUUUAAUGGAUACACGUCUCCUU)r-5′ | 127 |
| 07-25- | hEGFR- | 5′-r(GGAAAUUACCUAUGUGCAGAGGAAU)-3′ | 128 |
| 024 | 25-24 | 3′-(CCUUUAAUGGAUACACGUCUCCUUA)r-5′ | 129 |
| 07-25- | hEGFR- | 5′-r(GAAAUUACCUAUGUGCAGAGGAAUU)-3′ | 130 |
| 025 | 25-25 | 3′-(CUUUAAUGGAUACACGUCUCCUUAA)r-5′ | 131 |
| 07-25- | hEGFR- | 5′-r(UACCUAUGUGCAGAGGAAUUAUGAU)-3′ | 132 |
| 026 | 25-26 | 3′-(AUGGAUACACGUCUCCUUAAUACUA)r-5′ | 133 |
| 07-25- | hEGFR- | 5′-r(CCUAUGUGCAGAGGAAUUAUGAUCU)-3′ | 134 |
| 027 | 25-27 | 3′-(GGAUACACGUCUCCUUAAUACUAGA)r-5′ | 135 |
| 07-25- | hEGFR- | 5′-r(UGCAGAGGAAUUAUGAUCUUUCCUU)-3′ | 136 |
| 028 | 25-28 | 3′-(ACGUCUCCUUAAUACUAGAAAGGAA)r-5′ | 137 |
| 07-25- | hEGFR- | 5′-r(CAGAGGAAUUAUGAUCUUUCCUUCU)-3′ | 138 |
| 029 | 25-29 | 3′-(GUCUCCUUAAUACUAGAAAGGAAGA)r-5′ | 139 |
| 07-25- | hEGFR- | 5′-r(UCCUAUGCCUUAGCAGUCUUAUCUA)-3′ | 140 |
| 030 | 25-30 | 3′-(AGGAUACGGAAUCGUCAGAAUAGAU)r-5′ | 141 |
| 07-25- | hEGFR- | 5′-r(CCUAUGCCUUAGCAGUCUUAUCUAA)-3′ | 142 |
| 031 | 25-31 | 3′-(GGAUACGGAAUCGUCAGAAUAGAUU)r-5′ | 143 |
| 07-25- | hEGFR- | 5′-r(CCUUAGCAGUCUUAUCUAACUAUGA)-3′ | 144 |
| 032 | 25-32 | 3′-(GGAAUCGUCAGAAUAGAUUGAUACU)r-5′ | 145 |
| 07-25- | hEGFR- | 5′-r(GGGACAUAGUCAGCAGUGACUUUCU)-3′ | 146 |
| 033 | 25-33 | 3′-(CCCUGUAUCAGUCGUCACUGAAAGA)r-5′ | 147 |
| 07-25- | hEGFR- | 5′-r(UAGUCAGCAGUGACUUUCUCAGCAA)-3′ | 148 |
| 034 | 25-34 | 3′-(AUCAGUCGUCACUGAAAGAGUCGUU)r-5′ | 149 |
| 07-25- | hEGFR- | 5′-r(CCCGUAAUUAUGUGGUGACAGAUCA)-3′ | 150 |
| 035 | 25-35 | 3′-(GGGCAUUAAUACACCACUGUCUAGU)r-5′ | 151 |
| 07-25- | hEGFR- | 5′-r(CCGUAAUUAUGUGGUGACAGAUCAC)-3′ | 152 |
| 036 | 25-36 | 3′-(GGCAUUAAUACACCACUGUCUAGUG)r-5′ | 153 |
| 07-25- | hEGFR- | 5′-r(CGUCCGCAAGUGUAAGAAGUGCGAA)-3′ | 154 |
| 037 | 25-37 | 3-(GCAGGCGUUCACAUUCUUCACGCUU)r-5′ | 155 |
| 07-25- | hEGFR- | 5′-r(CAAGUGUAAGAAGUGCGAAGGGCCU)-3′ | 156 |
| 038 | 25-38 | 3′-(GUUCACAUUCUUCACGCUUCCCGGA)r-5′ | 157 |
| 07-25- | hEGFR- | 5′-r(CCUUGCCGCAAAGUGUGUAACGGAA)-3′ | 158 |
| 039 | 25-39 | 3′-(GGAACGGCGUUUCACACAUUGCCUU)r-5′ | 159 |
| 07-25- | hEGFR- | 5′-r(CCGCAAAGUGUGUAACGGAAUAGGU)-3′ | 160 |
| 040 | 25-40 | 3′-(GGCGUUUCACACAUUGCCUUAUCCA)r-5′ | 161 |
| 07-25- | hEGFR- | 5′-r(CGCAAAGUGUGUAACGGAAUAGGUA)-3′ | 162 |
| 041 | 25-41 | 3′-(GCGUUUCACACAUUGCCUUAUCCAU)r-5′ | 163 |
| 07-25- | hEGFR- | 5′-r(GCAAAGUGUGUAACGGAAUAGGUAU)-3′ | 164 |
| 042 | 25-42 | 3′-(CGUUUCACACAUUGCCUUAUCCAUA)r-5′ | 165 |
| 07-25- | hEGFR- | 5′-r(CAAAGUGUGUAACGGAAUAGGUAUU)-3′ | 166 |
| 043 | 25-43 | 3′-(GUUUCACACAUUGCCUUAUCCAUAA)r-5′ | 167 |
| 07-25- | hEGFR- | 5′-r(CACUCUCCAUAAAUGCUACGAAUAU)-3′ | 168 |
| 044 | 25-44 | 3′-(GUGAGAGGUAUUUACGAUGCUUAUA)r-5′ | 169 |
| 07-25- | hEGFR- | 5′-r(CCUCUGGAUCCACAGGAACUGGAUA)-3′ | 170 |
| 045 | 25-45 | 3′-(GGAGACCUAGGUGUCCUUGACCUAU)r-5′ | 171 |
| 07-25- | hEGFR- | 5′-r(UGGAUCCACAGGAACUGGAUAUUCU)-3′ | 172 |
| 046 | 25-46 | 3′-(ACCUAGGUGUCCUUGACCUAUAAGA)r-5′ | 173 |
| 07-25- | hEGFR- | 5′-r(UCCAUGCCUUUGAGAACCUAGAAAU)-3′ | 174 |
| 047 | 25-47 | 3′-(AGGUACGGAAACUCUUGGAUCUUUA)r-5′ | 175 |
| 07-25- | hEGFR- | 5′-r(CAGGACCAAGCAACAUGGUCAGUUU)-3′ | 176 |
| 048 | 25-48 | 3′-(GUCCUGGUUCGUUGUACCAGUCAAA)r-5′ | 177 |
| 07-25- | hEGFR- | 5′-r(UCUCUUGCAGUCGUCAGCCUGAACA)-3′ | 178 |
| 049 | 25-49 | 3′-(AGAGAACGUCAGCAGUCGGACUUGU)r-5′ | 179 |
| 07-25- | hEGFR- | 5′-r(CAUCCUUGGGAUUACGCUCCCUCAA)-3′ | 180 |
| 050 | 25-50 | 3′-(GUAGGAACCCUAAUGCGAGGGAGUU)r-5′ | 181 |
| 07-25- | hEGFR- | 5′-r(CCCUCAAGGAGAUAAGUGAUGGAGA)-3′ | 182 |
| 051 | 25-51 | 3′-(GGGAGUUCCUCUAUUCACUACCUCU)r-5′ | 183 |
| 07-25- | hEGFR- | 5′-r(CCUCAAGGAGAUAAGUGAUGGAGAU)-3′ | 184 |
| 052 | 25-52 | 3′-(GGAGUUCCUCUAUUCACUACCUCUA)r-5′ | 185 |
| 07-25- | hEGFR- | 5′-r(UCAAGGAGAUAAGUGAUGGAGAUGU)-3′ | 186 |
| 053 | 25-53 | 3′-(AGUUCCUCUAUUCACUACCUCUACA)r-5′ | 187 |
| 07-25- | hEGFR- | 5′-r(AGGAGAUAAGUGAUGGAGAUGUGAU)-3′ | 188 |
| 054 | 25-54 | 3′-(UCCUCUAUUCACUACCUCUACACUA)r-5′ | 189 |
| 07-25- | hEGFR- | 5′-r(GGAGAUAAGUGAUGGAGAUGUGAUA)-3′ | 190 |
| 055 | 25-55 | 3′-(CCUCUAUUCACUACCUCUACACUAU)r-5′ | 191 |
| 07-25- | hEGFR- | 5′-r(GAGAUAAGUGAUGGAGAUGUGAUAA)-3′ | 192 |
| 056 | 25-56 | 3′-(CUCUAUUCACUACCUCUACACUAUU)r-5′ | 193 |
| 07-25- | hEGFR- | 5′-r(CCAAGGGAGUUUGUGGAGAACUCUG)-3′ | 194 |
| 057 | 25-57 | 3′-(GGUUCCCUCAAACACCUCUUGAGAC)r-5′ | 195 |
| 07-25- | hEGFR- | 5′-r(GAGUUUGUGGAGAACUCUGAGUGCA)-3′ | 196 |
| 058 | 25-58 | 3′-(CUCAAACACCUCUUGAGACUCACGU)r-5′ | 197 |
| 07-25- | hEGFR- | 5′-r(CAGACAACUGUAUCCAGUGUGCCCA)-3′ | 198 |
| 059 | 25-59 | 3′-(GUCUGUUGACAUAGGUCACACGGGU)r-5′ | 199 |
| 07-25- | hEGFR- | 5′-r(UGUAUCCAGUGUGCCCACUACAUUG)-3′ | 200 |
| 060 | 25-60 | 3′-(ACAUAGGUCACACGGGUGAUGUAAC)r-5′ | 201 |
| 07-25- | hEGFR- | 5′-r(CAUCCAAACUGCACCUACGGAUGCA)-3′ | 202 |
| 061 | 25-61 | 3′-(GUAGGUUUGACGUGGAUGCCUACGU)r-5′ | 203 |
| 07-25- | hEGFR- | 5′-r(GCACGGUGUAUAAGGGACUCUGGAU)-3′ | 204 |
| 062 | 25-62 | 3′-(CGUGCCACAUAUUCCCUGAGACCUA)r-5′ | 205 |
| 07-25- | hEGFR- | 5′-r(CCGUCGCUAUCAAGGAAUUAAGAGA)-3′ | 206 |
| 063 | 25-63 | 3′-(GGCAGCGAUAGUUCCUUAAUUCUCU)r-5′ | 207 |
| 07-25- | hEGFR- | 5′-r(CGUCGCUAUCAAGGAAUUAAGAGAA)-3′ | 208 |
| 064 | 25-64 | 3′-(GCAGCGAUAGUUCCUUAAUUCUCUU)r-5′ | 209 |
| 07-25- | hEGFR- | 5′-r(CGCUAUCAAGGAAUUAAGAGAAGCA)-3′ | 210 |
| 065 | 25-65 | 3′-(GCGAUAGUUCCUUAAUUCUCUUCGU)r-5′ | 211 |
| 07-25- | hEGFR- | 5′-r(CAAGGAAUUAAGAGAAGCAACAUCU)-3′ | 212 |
| 066 | 25-66 | 3′-(GUUCCUUAAUUCUCUUCGUUGUAGA)r-5′ | 213 |
| 07-25- | hEGFR- | 5′-r(GAAAUCCUCGAUGAAGCCUACGUGA)-3′ | 214 |
| 067 | 25-67 | 3′-(CUUUAGGAGCUACUUCGGAUGCACU)r-5′ | 215 |
| 07-25- | hEGFR- | 5′-r(CCGCAGCAUGUCAAGAUCACAGAUU)-3′ | 216 |
| 068 | 25-68 | 3′-(GGCGUCGUACAGUUCUAGUGUCUAA)r-5′ | 217 |
| 07-25- | hEGFR- | 5′-r(CGCAGCAUGUCAAGAUCACAGAUUU)-3′ | 218 |
| 069 | 25-69 | 3′-(GCGUCGUACAGUUCUAGUGUCUAAA)r-5′ | 219 |
| 07-25- | hEGFR- | 5′-r(CAUGCAGAAGGAGGCAAAGUGCCUA)-3′ | 220 |
| 070 | 25-70 | 3′-(GUACGUCUUCCUCCGUUUCACGGAU)r-5′ | 221 |
| 07-25- | hEGFR- | 5′-r(CAGAAGGAGGCAAAGUGCCUAUCAA)-3′ | 222 |
| 071 | 25-71 | 3′-(GUCUUCCUCCGUUUCACGGAUAGUU)r-5′ | 223 |
| 07-25- | hEGFR- | 5′-r(AAGUGCCUAUCAAGUGGAUGGCAUU)-3′ | 224 |
| 072 | 25-72 | 3′-(UUCACGGAUAGUUCACCUACCGUAA)r-5′ | 225 |
| 07-25- | hEGFR- | 5′-r(GCCUAUCAAGUGGAUGGCAUUGGAA)-3′ | 226 |
| 073 | 25-73 | 3′-(CGGAUAGUUCACCUACCGUAACCUU)r-5′ | 227 |
| 07-25- | hEGFR- | 5′-r(CCUAUCAAGUGGAUGGCAUUGGAAU)-3′ | 228 |
| 074 | 25-74 | 3′-(GGAUAGUUCACCUACCGUAACCUUA)r-5′ | 229 |
| 07-25- | hEGFR- | 5′-r(CAAGUGGAUGGCAUUGGAAUCAAUU)-3′ | 230 |
| 075 | 25-75 | 3′-(GUUCACCUACCGUAACCUUAGUUAA)r-5′ | 231 |
| 07-25- | hEGFR- | 5′-r(CAGCCACCCAUAUGUACCAUCGAUG)-3′ | 232 |
| 076 | 25-76 | 3′-(GUCGGUGGGUAUACAUGGUAGCUAC)r-5′ | 233 |
| 07-25- | hEGFR- | 5′-r(CACCCAUAUGUACCAUCGAUGUCUA)-3′ | 234 |
| 077 | 25-77 | 3′-(GUGGGUAUACAUGGUAGCUACAGAU)r-5′ | 235 |
| 07-25- | hEGFR- | 5′-r(UGUACCAUCGAUGUCUACAUGAUCA)-3′ | 236 |
| 078 | 25-78 | 3′-(ACAUGGUAGCUACAGAUGUACUAGU)r-5′ | 237 |
| 07-25- | hEGFR- | 5′-r(UAGACGCAGAUAGUCGCCCAAAGUU)-3′ | 238 |
| 079 | 25-79 | 3′-(AUCUGCGUCUAUCAGCGGGUUUCAA)r-5′ | 239 |
| 07-25- | hEGFR- | 5′-r(CCAAGUCCUACAGACUCCAACUUCU)-3′ | 240 |
| 080 | 25-80 | 3′-(GGUUCAGGAUGUCUGAGGUUGAAGA)r-5′ | 241 |
| 07-25- | hEGFR- | 5′-r(CAAGUCCUACAGACUCCAACUUCUA)-3′ | 242 |
| 081 | 25-81 | 3′-(GUUCAGGAUGUCUGAGGUUGAAGAU)r-5′ | 243 |
| 07-25- | hEGFR- | 5′-r(ACUCCAACUUCUACCGUGCCCUGAU)-3′ | 244 |
| 082 | 25-82 | 3′-(UGAGGUUGAAGAUGGCACGGGACUA)r-5′ | 245 |
| 07-25- | hEGFR- | 5′-r(UCUCUGAGUGCAACCAGCAACAAUU)-3′ | 246 |
| 083 | 25-83 | 3′-(AGAGACUCACGUUGGUCGUUGUUAA)r-5′ | 247 |
| 07-25- | hEGFR- | 5′-r(CAAUUCCACCGUGGCUUGCAUUGAU)-3′ | 248 |
| 084 | 25-84 | 3′-(GUUAAGGUGGCACCGAACGUAACUA)r-5′ | 249 |
| 07-25- | hEGFR- | 5′-r(CCACCGUGGCUUGCAUUGAUAGAAA)-3′ | 250 |
| 085 | 25-85 | 3′-(GGUGGCACCGAACGUAACUAUCUUU)r-5′ | 251 |
| 07-25- | hEGFR- | 5′-r(CACCGUGGCUUGCAUUGAUAGAAAU)-3′ | 252 |
| 086 | 25-86 | 3′-(GUGGCACCGAACGUAACUAUCUUUA)r-5′ | 253 |
| 07-25- | hEGFR- | 5′-r(UGCAUUGAUAGAAAUGGGCUGCAAA)-3′ | 254 |
| 087 | 25-87 | 3′-(ACGUAACUAUCUUUACCCGACGUUU)r-5′ | 255 |
| 07-25- | hEGFR- | 5′-r(GCAUUGAUAGAAAUGGGCUGCAAAG)-3′ | 256 |
| 088 | 25-88 | 3′-(CGUAACUAUCUUUACCCGACGUUUC)r-5′ | 257 |
| 07-25- | hEGFR- | 5′-r(CAGCUUCUUGCAGCGAUACAGCUCA)-3′ | 258 |
| 089 | 25-89 | 3′-(GUCGAAGAACGUCGCUAUGUCGAGU)r-5′ | 259 |
| 07-25- | hEGFR- | 5′-r(CCAGUGCCUGAAUACAUAAACCAGU)-3′ | 260 |
| 090 | 25-90 | 3′-(GGUCACGGACUUAUGUAUUUGGUCA)r-5′ | 261 |
| 07-25- | hEGFR- | 5′-r(CCACCUGUGUCAACAGCACAUUCGA)-3′ | 262 |
| 091 | 25-91 | 3′-(GGUGGACACAGUUGUCGUGUAAGCU)r-5′ | 263 |
| 07-25- | hEGFR- | 5′-r(GCCCAGAAAGGCAGCCACCAAAUUA)-3′ | 264 |
| 092 | 25-92 | 3′-(CGGGUCUUUCCGUCGGUGGUUUAAU)r-5′ | 265 |
| 07-25- | hEGFR- | 5′-r(CCCAGAAAGGCAGCCACCAAAUUAG)-3′ | 266 |
| 093 | 25-93 | 3′-(GGGUCUUUCCGUCGGUGGUUUAAUC)r-5′ | 267 |
| 07-25- | hEGFR- | 5′-r(GGAAGCCAAGCCAAAUGGCAUCUUU)-3′ | 268 |
| 094 | 25-94 | 3′-(CCUUCGGUUCGGUUUACCGUAGAAA)r-5′ | 269 |
| 07-25- | hEGFR- | 5′-r(GAAGCCAAGCCAAAUGGCAUCUUUA)-3′ | 270 |
| 095 | 25-95 | 3′-(CUUCGGUUCGGUUUACCGUAGAAAU)r-5′ | 271 |
| 07-25- | hEGFR- | 5′-r(AAGCCAAGCCAAAUGGCAUCUUUAA)-3′ | 272 |
| 096 | 25-96 | 3′-(UUCGGUUCGGUUUACCGUAGAAAUU)r-5′ | 273 |
| 07-25- | hEGFR- | 5′-r(UGGCAUCUUUAAGGGCUCCACAGCU)-3′ | 274 |
| 097 | 25-97 | 3′-(ACCGUAGAAAUUCCCGAGGUGUCGA)r-5′ | 275 |
| 07-25- | hEGFR- | 5′-r(CAUCUUUAAGGGCUCCACAGCUGAA)-3′ | 276 |
| 098 | 25-98 | 3′-(GUAGAAAUUCCCGAGGUGUCGACUU)r-5′ | 277 |
| 07-25- | hEGFR- | 5′-r(CCACGGAGGAUAGUAUGAGCCCUAA)-3′ | 278 |
| 099 | 25-99 | 3′-(GGUGCCUCCUAUCAUACUCGGGAUU)r-5′ | 279 |
| 07-25- | hEGFR- | 5′-r(CACGGAGGAUAGUAUGAGCCCUAAA)-3′ | 280 |
| 100 | 25-100 | 3′-(GUGCCUCCUAUCAUACUCGGGAUUU)r-5′ | 281 |
| 07-25- | hEGFR- | 5′-r(UACAGAAACGCAUCCAGCAAGAAUA)-3′ | 282 |
| 101 | 25-101 | 3′-(AUGUCUUUGCGUAGGUCGUUCUUAU)r-5′ | 283 |
| 07-25- | hEGFR- | 5′-r(UGAUGGACCAGUGGUUUCCAGUCAU)-3′ | 284 |
| 102 | 25-102 | 3′-(ACUACCUGGUCACCAAAGGUCAGUA)r-5′ | 285 |
| 07-25- | hEGFR- | 5′-r(CAGUGGUUUCCAGUCAUGAGCGUUA)-3′ | 286 |
| 103 | 25-103 | 3′-(GUCACCAAAGGUCAGUACUCGCAAU)r-5′ | 287 |
| 07-25- | hEGFR- | 5′-r(CAGCAAGAGAGGAUGACACAUCAAA)-3′ | 288 |
| 104 | 25-104 | 3′-(GUCGUUCUCUCCUACUGUGUAGUUU)r-5′ | 289 |
| 07-25- | hEGFR- | 5′-r(CCAGCCCACAUUGGAUUCAUCAGCA)-3′ | 290 |
| 105 | 25-105 | 3′-(GGUCGGGUGUAACCUAAGUAGUCGU)r-5′ | 291 |
| 07-25- | hEGFR- | 5′-r(CAGCCCACAUUGGAUUCAUCAGCAU)-3′ | 292 |
| 106 | 25-106 | 3′-(GUCGGGUGUAACCUAAGUAGUCGUA)r-5′ | 293 |
| 07-25- | hEGFR- | 5′-r(GCCCACAUUGGAUUCAUCAGCAUUU)-3′ | 294 |
| 107 | 25-107 | 3′-(CGGGUGUAACCUAAGUAGUCGUAAA)r-5′ | 295 |
| 07-25- | hEGFR- | 5′-r(CCACAGCUGAGAAUGUGGAAUACCU)-3′ | 296 |
| 108 | 25-108 | 3′-(GGUGUCGACUCUUACACCUUAUGGA)r-5′ | 297 |
| 07-25- | hEGFR- | 5′-r(CACAGCUGAGAAUGUGGAAUACCUA)-3′ | 298 |
| 109 | 25-109 | 3′-(GUGUCGACUCUUACACCUUAUGGAU)r-5′ | 299 |
| 07-25- | hEGFR- | 5′-r(UCUCCUAAUUUGAGGCUCAGAUGAA)-3′ | 300 |
| 110 | 25-110 | 3′-(AGAGGAUUAAACUCCGAGUCUACUU)r-5′ | 301 |
| 07-25- | hEGFR- | 5′-r(GAGGCUCAGAUGAAAUGCAUCAGGU)-3′ | 302 |
| 111 | 25-111 | 3′-(CUCCGAGUCUACUUUACGUAGUCCA)r-5′ | 303 |
| 07-25- | hEGFR- | 5′-r(CAGGUGCGAAUGACAGUAGCAUUAU)-3′ | 304 |
| 112 | 25-112 | 3′-(GUCCACGCUUACUGUCAUCGUAAUA)r-5′ | 305 |
| 07-25- | hEGFR- | 5′-r(GCGAAUGACAGUAGCAUUAUGAGUA)-3′ | 306 |
| 113 | 25-113 | 3′-(CGCUUACUGUCAUCGUAAUACUCAU)r-5′ | 307 |
| 07-25- | hEGFR- | 5′-r(CAGUAGCAUUAUGAGUAGUGUGGAA)-3′ | 308 |
| 114 | 25-114 | 3′-(GUCAUCGUAAUACUCAUCACACCUU)r-5′ | 309 |
| 07-25- | hEGFR- | 5′-r(GCAUUAUGAGUAGUGUGGAAUUCAG)-3′ | 310 |
| 115 | 25-115 | 3′-(CGUAAUACUCAUCACACCUUAAGUC)r-5′ | 311 |
| 07-25- | hEGFR- | 5′-r(AGUAGUGUGGAAUUCAGGUAGUAAA)-3′ | 312 |
| 116 | 25-116 | 3′-(UCAUCACACCUUAAGUCCAUCAUUU)r-5′ | 313 |
| 07-25- | hEGFR- | 5′-r(UGUGCCCUGUAACCUGACUGGUUAA)-3′ | 314 |
| 117 | 25-117 | 3′-(ACACGGGACAUUGGACUGACCAAUU)r-5′ | 315 |
| 07-25- | hEGFR- | 5′-r(CCUGACUGGUUAACAGCAGUCCUUU)-3′ | 316 |
| 118 | 25-118 | 3′-(GGACUGACCAAUUGUCGUCAGGAAA)r-5′ | 317 |
| 07-25- | hEGFR- | 5′-r(GACUGGUUAACAGCAGUCCUUUGUA)-3′ | 318 |
| 119 | 25-119 | 3′-(CUGACCAAUUGUCGUCAGGAAACAU)r-5′ | 319 |
| 07-25- | hEGFR- | 5′-r(CAGCAGUCCUUUGUAAACAGUGUUU)-3′ | 320 |
| 120 | 25-120 | 3′-(GUCGUCAGGAAACAUUUGUCACAAA)r-5′ | 321 |
| 07-25- | hEGFR- | 5′-r(CAGCCUACAGUUAUGUUCAGUCACA)-3′ | 322 |
| 121 | 25-121 | 3′-(GUCGGAUGUCAAUACAAGUCAGUGU)r-5′ | 323 |

The candidate siRNA molecules described in this Example can be used for inhibition of expression of hEGFR and are useful in a variety of therapeutic settings, for example, in the treatment of cardiovascular disorders such as aortic valve disease and cancers including but not limited to breast, lung, prostate, colorectal, brain, esophageal, bladder, pancreatic, cervical, head and neck, meningioma, kidney, endometrial, and ovarian cancer, melanoma, lymphoma, glioblastoma, multidrug resistant cancers, and any other cancerous diseases, and/or other disease states, conditions, or traits associated with hEGFR gene expression or activity in a subject or organism.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A nucleic acid molecule that down regulates expression of an epidermal growth factor receptor (EGFR) gene, wherein the nucleic acid molecule comprises a nucleic acid that targets any one of the polynucleotide sequences set forth in SEQ ID NOs: 1-10 or 21-121.

2. The nucleic acid molecule of claim 1, wherein the nucleic acid is a short interfering RNA (siRNA) molecule.

3. The nucleic acid of claim 2, wherein the siRNA comprises any one of the single stranded RNA sequences provided in SEQ ID NOs: 11-20 and 122-323, or a double-stranded RNA thereof.

4. The nucleic acid molecule of claim 1, wherein the nucleic acid molecule down regulates expression of an EGFR gene via RNA interference (RNAi).

5. A composition comprising any one or more of the siRNA molecules of claim 3.

6. The composition of claim 5 further comprising a targeting moiety.

7. The composition of claim 5 further comprising a histidine-lysine copolymer.

8. A method for treating or preventing a cancer in a subject with an EGFR-expressing cancer and having or suspected of being at risk for having the cancer, comprising administering to the subject the composition of claim 5, thereby treating or preventing the cancer.

9. The method of claim 8 wherein the cancer is selected from the group consisting of breast cancer, lung cancer, prostate cancer, colorectal cancer, brain cancer, esophageal cancer, stomach cancer, bladder cancer, pancreatic cancer, cervical cancer, head and neck cancer, kidney cancer, endometrial cancer, ovarian cancer, meningioma, melanoma, lymphoma, and glioblastoma.

10. A method for reducing the synthesis or expression of EGFR in a cell, comprising introducing into the cell one or more siRNAs, wherein the one or more siRNAs have a sequence as set forth in SEQ ID NOs: 11-20 or 122-323.
